(19) United States
(12) Patent Application Publication
(10) Pub. No.: US 2023/0024863 A1
(43) Pub. Date: Jan. 26, 2023

(54) ENDOPROSTHETIC ROTATING HINGE KNEE ASSEMBLIES, SUBASSEMBLIES, AND METHODS

(71) Applicant: MicroPort Orthopedics Holdings Inc., Arlington, TN (US)

(72) Inventors: Fred W. Bowman, Germantown, TN (US); Kristen F. Mountjoy, Allendale, NJ (US); Michael L. Brooks, Brighton, TN (US); Jacob H. Carroll, Oakland, TN (US)

(73) Assignee: MicroPort Orthopedics Holdings Inc., Arlington, TN (US)

(21) Appl. No.: 17/660,252

(22) Filed: Apr. 22, 2022

Related U.S. Application Data
(60) Provisional application No. 63/225,109, filed on Jul. 23, 2021.

(51) Int. Cl. A61F 2/38

(52) U.S. Cl. CPC A61F 2/385; A61F 2/389; A61F 2/3868; A61F 2220/0025

(57) ABSTRACT

Assemblies, systems, kits, and methods related to an endoprosthetic rotating hinge assembly. The exemplary embodiments disclosed herein can comprise a preassembled rotating hinge subassembly having a hingedly rotating femur box configured to be mechanically engaged to a femoral component via a femur fastening mechanism, the femur fastening mechanism being non-axially aligned with a tibial axis of rotation when the knee is in flexion or extension.

### REFERENCE TO RELATED APPLICATION

This application claims the benefit of U.S. Provisional Application No. 63/225,109 filed on Jul. 23, 2021. The disclosure of this related application is hereby incorporated into this disclosure in its entirety.

### BACKGROUND OF THE INVENTION

### 1. Technical Field

The present disclosure relates generally to the field of knee implants, and more particularly to rotating hinge implants for revision and primary knees and methods of implantation thereof.

### 2. Related Art

Rotating hinged knee implants are typically used in revision knee surgeries in situations where the natural ligaments and other retaining anatomy of the patient's knee are severely compromised. Non-rotating hinged knee implants were initially used to replace the stabilizing function of a missing posterior cruciate ligament (“PCL”). However, surgeons recognized early on that simple hinge knees were prone to accelerated wear and failure. This problem was addressed by providing a rotating hinge function, such that the tibia rotates around a generally vertical tibial axis relative to the femur during flexion. Several designs have been used for this purpose. See e.g., U.S. Pat. Nos. 6,773,461 and 10,682,236.

Some drawbacks of conventional rotating hinge knees include: the hinge pin is often inserted from the lateral side, which requires multiple incisions into the leg. Some hinge rotating assemblies, like the one disclosed in U.S. Pat. No. 6,773,461 must be assembled intraoperatively. Such designs increase the duration of the procedure. Increased procedure duration likewise increases the risk of infection and other complications attributable to time under anesthesia. Other designs, such as the rotating hinge assembly disclosed in U.S. Pat. No. 10,682,236, require multiple locking mechanisms. Such mechanism also increase installation and overall procedure time. Complicated locking mechanisms can also be difficult to undo in the event that the patient undergoes a future revision surgery.

Furthermore, the devices disclosed in both U.S. Pat. Nos. 6,773,461 and 10,682,236 have retaining elements that are inserted and disposed around the tibial rotational axis. In operation, transverse load-bearing elements such as screw threads that are disposed around the tibial rotational axis generally experience significant compressive forces from the femur. This, coupled with torsional forces that the screw threads may experience during normal bending and rotation of the knee joint, may cause these threaded elements (or their associated parts) to fail prematurely. Premature implant failure can result in an otherwise avoidable further revision surgery to fix or replace the implant. Replacing the entire implant often involves cutting away the bone into which the implant was attached. Rotating hinge assemblies are frequently used on patients that already suffer from significant bone degradation. Further revising a failed rotating hinge assembly may not be possible in certain cases if insufficient bone remains.

### SUMMARY

There is therefore a need for an improved rotating hinge knee having the properties, characteristics and functionality described herein.

The problem of lengthy and cumbersome surgical installation procedure and the problems of rotating hinge implants failing prematurely due to mechanical wear of internal tibial aligned implant fastening elements can be mitigated by an exemplary knee joint endoprosthetic assembly comprising: a rotating hinge subassembly having a femur box that can hingedly articulate around a tibial yoke (e.g., via a transverse hinge pin), wherein the femur box is configured to be mechanically engaged to a femoral component via a femur fastener, the femur fastener being non-axially aligned with a tibial axis of rotation when the knee is in flexion or extension, and wherein the transverse hinge pin of the rotating hinge subassembly does not mechanically engage the femoral component in an installed configuration.

It is contemplated that certain exemplary embodiments disclosed herein may allow for implantation of a rotating hinge knee through a single incision.

It is further contemplated that certain exemplary embodiments in accordance with this disclosure may provide a rotating hinge knee having a preassembled rotating hinge subassembly that is not preassembled with the femoral component or the tibial component of the endoprosthetic implant.

It is still further contemplated that certain exemplary embodiments in accordance with the present disclosure may provide a rotating hinge knee having one femur fastener for securing the rotating hinge subassembly to the femoral component of the endoprosthesis, wherein the femur fastener engages the femoral component in a parasagittal plane.

It is yet still further contemplated that certain exemplary embodiments in accordance with this disclosure may disallow the need for excessive dislocation of the distal femur relative to the proximal tibia during endoprosthetic implant installation and assembly, which may minimize resections of surrounding soft tissue and thereby contribute to faster patient recovery.

Still further exemplary embodiments disclosed herein may provide a rotating hinge knee having an extension stop for interaction with the femoral component.

The foregoing objectives are achieved by providing a rotating hinge knee implant assembly having the features described herein.

### BRIEF DESCRIPTION OF THE DRAWINGS

The foregoing will be apparent from the following more particular description of exemplary embodiments of the disclosure, as illustrated in the accompanying drawings. The drawings are not necessarily to scale, with emphasis instead being placed upon illustrating the disclosed embodiments.

FIG. 1 is front view of an exemplary embodiment of an endoprosthetic rotating hinge knee implant assembly in an assembled state (i.e., an installed configuration).

FIG. 2A is an anterior perspective view of one exemplary embodiment of a femoral component and a tibial component of an endoprosthetic rotating hinge knee implant assembly.

FIG. 2B shows an anterior perspective view of one exemplary embodiment of a rotating hinge subassembly for use in an endoprosthetic rotating hinge knee implant assembly.

FIG. 2C shows an anterior perspective view of one exemplary embodiment of a rotating hinge knee implant assembly showing insertion of a rotating hinge subassembly into the tibial component.

FIG. 2D shows an anterior perspective view of one exemplary embodiment of an endoprosthetic rotating hinge knee implant assembly showing attachment of a rotating hinge subassembly to the femoral component.

FIG. 3A shows a cross-sectional lateral view of one exemplary embodiment of an endoprosthetic rotating hinge knee implant assembly in extension; the cross section is taken from a parasagittal plane that bisects the exemplary endoprosthetic rotating hinge implant assembly.

FIG. 3B shows a cross-sectional lateral view of one exemplary embodiment of an endoprosthetic rotating hinge knee implant assembly in flexion; the cross section is taken from a parasagittal plane that bisects the exemplary endoprosthetic rotating hinge implant assembly.

FIG. 4 shows an exploded view of one embodiment of an exemplary endoprosthetic rotating hinge knee implant assembly and an exploded view of an exemplary rotating hinge knee subassembly.

FIG. 5 shows a perspective view of one embodiment of a femur box for use in an exemplary rotating hinge subassembly.

FIG. 6 shows a perspective view of one embodiment of a femur box bearing member for use with an exemplary endoprosthetic rotating hinge knee implant assembly.

FIG. 7A shows a lateral view of one embodiment of an exemplary tibial yoke for use in an endoprosthetic rotating hinge subassembly.

FIG. 7B depicts an anterior view of the exemplary embodiment of the tibial yoke depicted in FIG. 7A.

FIG. 7C depicts a top down view of the exemplary embodiment of the tibial yoke depicted in FIGS. 7A and 7B.

FIG. 7D is a bottom-up view of the exemplary embodiment of the tibial yoke depicted in FIGS. 7A, 7B, and 7C.

FIG. 8A shows a top-side perspective view of one embodiment of a modular extension stop for use in a rotating hinge subassembly.

FIG. 8B shows a lateral view of one embodiment of a modular extension stop for use in a rotating hinge subassembly.

FIG. 8C shows an anterior view of one embodiment of a modular extension stop for use in a rotating hinge subassembly.

FIG. 9 shows a perspective leading end to trailing end view of one embodiment of a femur fastener for use with a rotating hinge knee implant assembly.

FIG. 10A shows a top-side perspective view of another embodiment of an extension stop for use with a rotating hinge knee implant assembly.

FIG. 10B shows a lateral view of another embodiment of a modular extension stop for use with a rotating hinge knee implant assembly.

FIG. 10C shows an anterior view of another embodiment of a modular extension stop for use with a rotating hinge knee implant assembly.

FIG. 11A is a perspective view of an exemplary endoprosthetic rotating hinge knee implant assembly showing the rotating hinge subassembly prior to being affixed to the femoral component via a femur fastener.

FIG. 11B is a perspective view of an exemplary endoprosthetic rotating hinge knee implant assembly showing the rotating hinge subassembly in an engaged position.

FIG. 12 is a cross-sectional lateral view of exemplary embodiment of a rotating hinge knee implant assembly in extension showing an alternative modular extension stop; the cross section is taken from a parasagittal plane that bisects the exemplary endoprosthetic rotating hinge implant assembly.

FIG. 13 is a schematic perspective view of anatomical planes relative to a person.

### DETAILED DESCRIPTION OF THE PREFERRED EMBODIMENTS

The following detailed description of the preferred embodiments is presented only for illustrative and descriptive purposes and is not intended to be exhaustive or to limit the scope and spirit of the invention. The embodiments were selected and described to best explain the principles of the invention and its practical application. One of ordinary skill in the art will recognize that many variations can be made to the invention disclosed in this specification without departing from the scope and spirit of the invention.

Similar reference characters indicate corresponding parts throughout the several views unless otherwise stated. Although the drawings represent embodiments of various features and components according to the present disclosure, the drawings are not necessarily to scale and certain features may be exaggerated to better illustrate embodiments of the present disclosure, and such exemplifications are not to be construed as limiting the scope of the present disclosure.

Except as otherwise expressly stated herein, the following rules of interpretation apply to this specification: (a) all words used herein shall be construed to be of such gender or number (singular or plural) as such circumstances require; (b) the singular terms “a,” “an,” and “the,” as used in the specification and the appended claims include plural references unless the context clearly dictates otherwise; (c) the antecedent term “about” applied to a recited range or value denotes an approximation with the deviation in the range or values known or expected in the art from the measurements; (d) the words, “herein,” “hereby,” “hereto,” “hereinbefore,” and “hereinafter,” and words of similar import, refer to this specification in its entirety and not to any particular paragraph, claim, or other subdivision, unless otherwise specified; (e) descriptive headings are for convenience only and shall not control or affect the meaning of construction of part of the specification; and (f) “or” and “any” are not exclusive and “include” and “including” are not limiting. Further, the terms, “comprising,” “having,” “including,” and “containing” are to be construed as open-ended terms (i.e., meaning “including but not limited to”).

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments, whether explicitly described.

To the extent necessary to provide descriptive support, the subject matter and/or text of the appended claims are incorporated herein by reference in their entirety.

Recitation of ranges of values herein are merely intended to serve as a shorthand method of referring individually to each separate value falling within the range of any sub-ranges there between, unless otherwise clearly indicated herein. Each separate value within a recited range is incorporated into the specification or claims as if each separate value were individually recited herein. Where a specific range of values is provided, it is understood that each intervening value, to the tenth or less of the unit of the lower limit between the upper and lower limit of that range and any other stated or intervening value in that stated range of sub range thereof, is included herein unless the context clearly dictates otherwise. All subranges are also included. The upper and lower limits of these smaller ranges are also included therein, subject to any specifically and expressly excluded limit in the stated range.

It should be noted that some of the terms used herein are relative terms. For example, the terms, “upper” and, “lower” are relative to each other in location, i.e., an upper component is located at a higher elevation than a lower component in each orientation, but these terms can change if the orientation is flipped. The terms, “inlet” and “outlet” are relative to the fluid flowing through them with respect to a given structure, e.g., a fluid flows through the inlet into the structure and then flows through the outlet out of the structure. The terms, “upstream” and “downstream” are relative to the direction in which a fluid flows through various components prior to flowing through the downstream component.

The terms, “horizontal” and “vertical” are used to indicate direction relative to an absolute reference, i.e., ground level. However, these terms should not be construed to require structure to be absolutely parallel or absolutely perpendicular to each other. For example, a first vertical structure and a second vertical structure are not necessarily parallel to each other. The terms, “top” and “bottom” or “base” are used to refer to locations or surfaces where the top is always higher than the bottom or base relative to an absolute reference, i.e., the surface of the Earth. The terms, “upwards” and “downwards” are also relative to an absolute reference; an upwards flow is always against the gravity of the Earth.

Throughout this disclosure, various positional terms, such as “distal,” “proximal,” “medial,” “lateral,” “anterior,” and “posterior,” will be used in the customary manner when referring to the human anatomy. More specifically, “distal” refers to the area away from the point of attachment to the body, while “proximal” refers to the area near the point of attachment to the body. For example, the distal femur refers to the portion of the femur near the tibia, whereas the proximal femur refers to the portion of the femur near the hip. The terms, “medial” and “lateral” are also essentially opposites. “Medial” refers to something that is disposed closer to the middle of the body. “Lateral” means that something is disposed closer to the right side or the left side of the body than to the middle of the body. Regarding, “anterior” and “posterior,” “anterior” refers to something disposed closer to the front of the body, whereas “posterior” refers to something disposed closer to the rear of the body.”

“Varus” and “valgus” are broad terms and include without limitation, rotational movement in a medial and/or lateral direction relative to the knee joint.

The phrase, “mechanical axis of the femur” refers to an imaginary line drawn from the center of the femoral head to the center of the distal femur at the knee.

The phrase, “mechanical axis of the tibia” refers to an imaginary line drawn from the center of the proximal tibia to the center of the distal tibia, which is just above the ankle.

The term, “anatomic axis” refers to an imaginary line drawn lengthwise down the middle of femoral shaft or tibial shaft, depending upon use.

Described herein are hinge knee assemblies, systems, and methods that may be configured to be implanted in a matter similar to currently available primary and revision surgical techniques.

During primary or revision knee surgeries, the surgeon generally makes a vertical midline incision on the anterior side of the operative knee. The incision is generally made with the knee in flexion at or below the tibial tuberosity and may extend several inches above the patella.

In a primary TKA, the surgeon continues to incise the fatty tissue to expose the anterior aspect of the joint capsule. The surgeon may then perform a medial parapatellar arthrotomy to pierce the joint capsule and resect the medial patellar retinaculum. A retractor is then commonly used to move the patella generally laterally to expose the distal condyles of the femur and the cartilaginous meniscus resting on the proximal tibial plateau. The surgeon then removes the meniscus and uses instrumentation to measure and resect the distal femur and proximal tibia to accommodate trial implants. Trial implants are test endoprostheses that generally have the same functional dimensions of the actual endoprostheses, but trial implants are designed to be temporarily installed and removed for the purposes of evaluating the fit of the actual endoprostheses and for the purposes of evaluating the knee joint's kinematics. The surgeon removes the trial implants and installs the actual implants once the surgeon is satisfied with the trial implant's sizing and the knee joint's kinematics.

In a revision procedure, after the surgeon has performed the initial incision, the surgeon may release scar tissue around the patellar tendon. The surgeon then moves (i.e., preforms a subluxation of) the patella or patellar implant generally laterally to expose the prior-installed implant, which usually has a femoral component installed on the distal femur, a tibial component installed on the proximal tibia, and a meniscal insert disposed between the femoral component and the tibial component. The surgeon then removes the prior-installed implant.

It will be appreciated that the type of prior-installed implant can vary from case to case. Prior-installed implants can include static spacers that have been inserted into aligned intramedullary bores in the distal femur and proximal tibia to immobilize the knee joint, or complex implants that have been used to reconstruct portions of the knee joint that had undergone trauma. However, common prior-installed implants include implants installed during a primary TKA, or prior revision implants. A variety of factors influence the decision to replace a prior-installed implant with a rotating hinge knee implant, but common factors include excessive wear or inoperability, the presence of trauma, bone degenerative disease progression, the integrity of the underlying bone, and severe varus/valgus deformities. Common bone degenerative diseases include rheumatoid arthritis and arthrosis.

Prior installed implants may have been bonded to bone in a variety of ways. Press-fit implants typically have a porous roughened surface on the connective side of the femoral and tibial components. The porous surface permits regrowth of the bone into these pours over time. Another very common bonding technique involves the use of an antibiotic infused grout commonly known as “bone cement” by people in the orthopedic industry. It will be appreciated that “bone cement” is a term of art even though bone cements themselves generally do not have adhesive properties. Bone cements generally rely on a close mechanical interlock between the irregular surface of the bone and the surface of the connective side of the endoprosthesis. Common bone cements include polymethyl methacrylate (“PMMA”), calcium phosphate cements (“CPCs”), and glass polyalkenoate isomer cements (“GPICs”).

Removal of the prior-installed implants generally involves cutting away the bone underlying the bone cement—or in the case of press-fit implants, the bone underlying the press-fit implant. This resection exposes fresh bone capable of receiving a revision press-fit or bone cement bondable implant. Removing bone to remove the prior-installed implant would move the joint line if the revision implant was not sized to replace the newly resected bone.

This tibial resection is usually coplanar with a transverse body plane that is perpendicular to the anatomic axis of the tibia. Once resected, the resected area of the tibia can be known as the “tibial plateau.” After the prior-installed implant has been removed, a series of differently sized intramedullary reamers may then be used to prepare or extend an intramedullary bore in both the tibia and the femur to seat the tibial and femoral components of the revision implant respectively. After reaming, the surgeon may use instrumentation to further measure and resect the proximal tibial plateau. Next, the surgeon may place a trial tibial component on the resected proximal tibial plateau. The surgeon generally uses different instrumentation to measure and resect the distal femoral condyles for the purpose of installing a trial femoral component. If the trial components are not seated appropriately, the surgeon may use further instrumentation to measure and resect the femoral condyles and/or the tibial plateau until the desired seating is achieved.

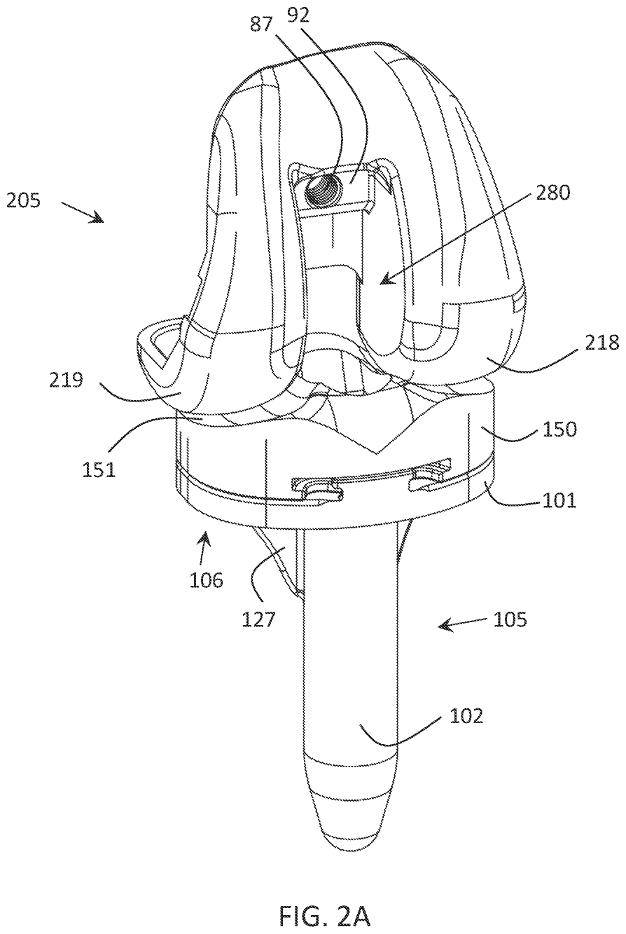

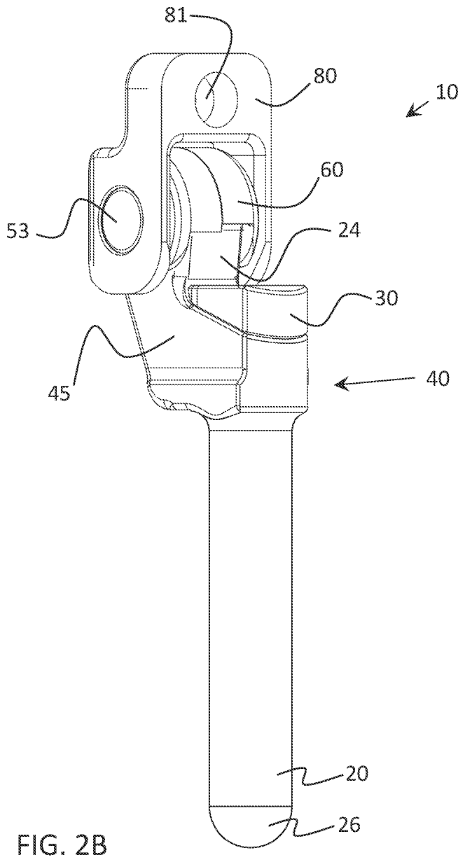

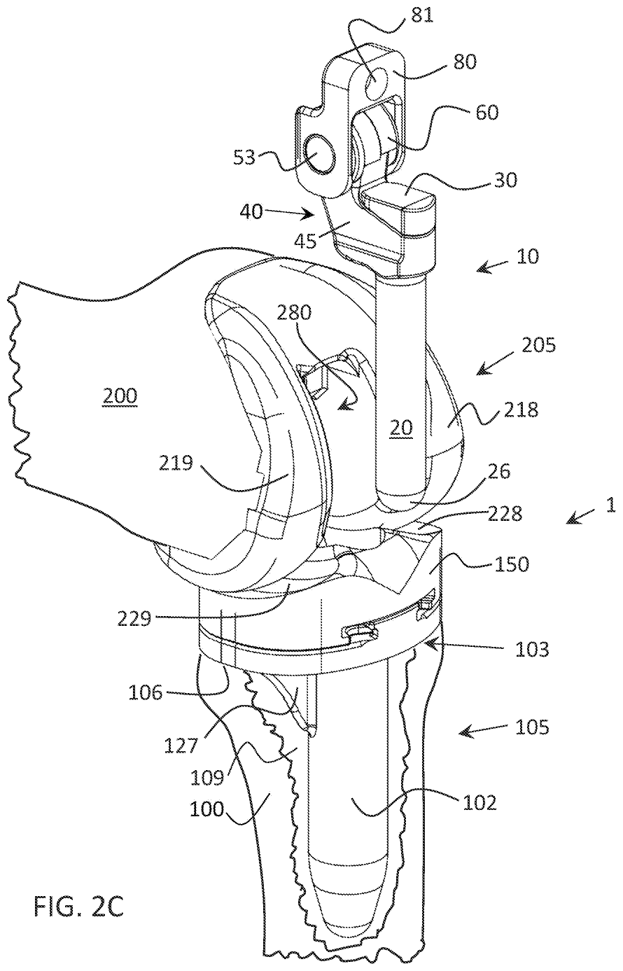

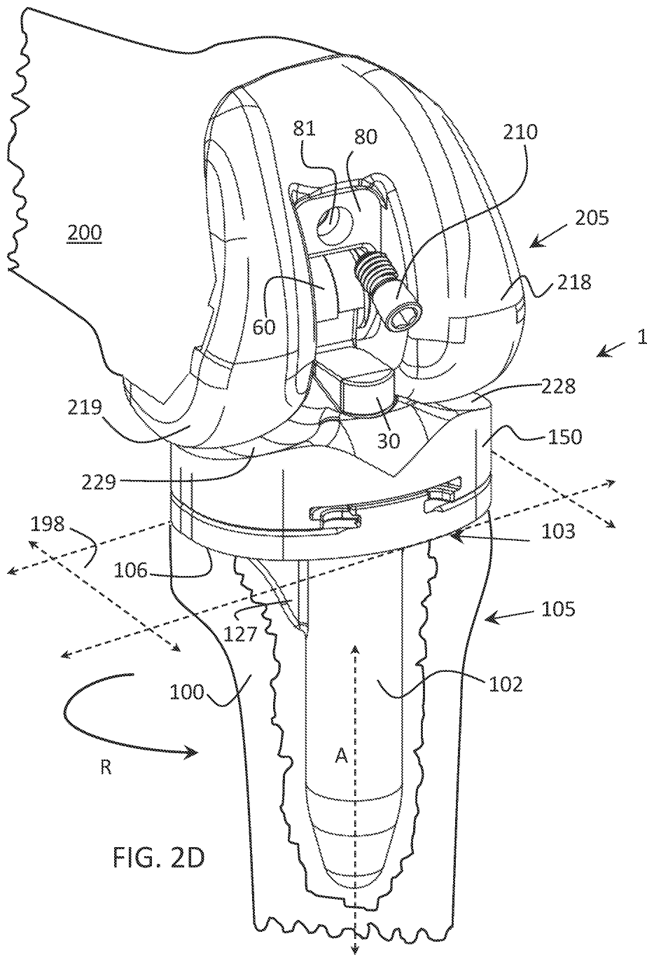

Rotating hinge knees typically have a femur box cavity (see 280, FIG. 2A) in the femoral component (see 205, FIG. 2A) of the endoprosthetic implant. Further resection of the lateral side of the medial condyle and the medial side of the lateral condyle is generally required to accommodate the femur box cavity. Stated differently, the surgeon makes a box-shaped cut out in the middle of the distal femur to accommodate the femur box cavity of the femoral component of the implant. Failure to properly resect the distal femur could result in intercondylar fractures.

The surgeon then generally inserts a trial meniscal insert between the trial tibial tray and the trial femoral component to test the knee's flexion and extension, general stability, and patellar tracking on the trial implants. Metallic blocks called “augments” can be attached to the tibial or femoral components to replace missing or compromised bone. Once satisfied with the trial and movement characteristics, the surgeon can use new uncured antibiotic infused bone cement to permanently affix the actual tibial and femoral components of the endoprosthetic implant or use a press-fit implant and avoid use of bone cement if desired.

Other rotating hinge knee systems differ from the present disclosure in that many other rotating hinge knee systems require intraoperative lateral assembly of the hinge component. This interoperative assembly requires additional resection of the lateral side of one or more femoral condyles to provide access to a hinge component (which is commonly a pin). Some designs require additional posterior resection of the condyles. Additional resections prolong the procedure and reduce the amount of remaining bone available to future revisions. It will also be appreciated that additional but unnecessary lateral and/or posterior condylar resections can prolong the patient's recovery time, introduce new areas that may become infected, and introduce an area of structural weakness—especially in cases with existing bone degradation. Any area of structural weakness increases the risk of catastrophic implant failure during normal use.

Interoperative assembly of a rotating hinge knee also prolongs the procedure and increases the risk of infection and other complications borne from increased time under anesthesia.

Some other rotating hinge knees use retaining elements having screw threads configured to be inserted and disposed around the tibial rotational axis. Without being bound by theory, it is contemplated that in operation, transverse load-bearing elements that are disposed around the tibial rotational axis generally experience significant compressive forces from the femur. This, coupled with torsional forces that the screw threads may experience during normal bending and rotation of the knee joint, may cause parts with these threaded elements to fail prematurely, thereby necessitating further revision surgeries to replace either the worn part or the entire implant.

Further, other rotating hinge knees are not configured to restrict five of the six degrees of freedom of the femoral component and the tibial component during installation. It is contemplated that the exemplary embodiments disclosed herein can further facilitate the installation process by permitting the surgeon to align the femoral component with the rotating hinge subassembly (see 10, FIG. 2C) without having to focus on the potential rotation of the femur or tibia during the alignment process.

To reduce installation time compared to existing rotating hinge knees, exemplary embodiments of the rotating hinge subassembly 10 described herein (see e.g., FIG. 2C) can come preassembled in a self-contained module that is configured to be mechanically affixed to the femoral component using a single femur fastener 210.

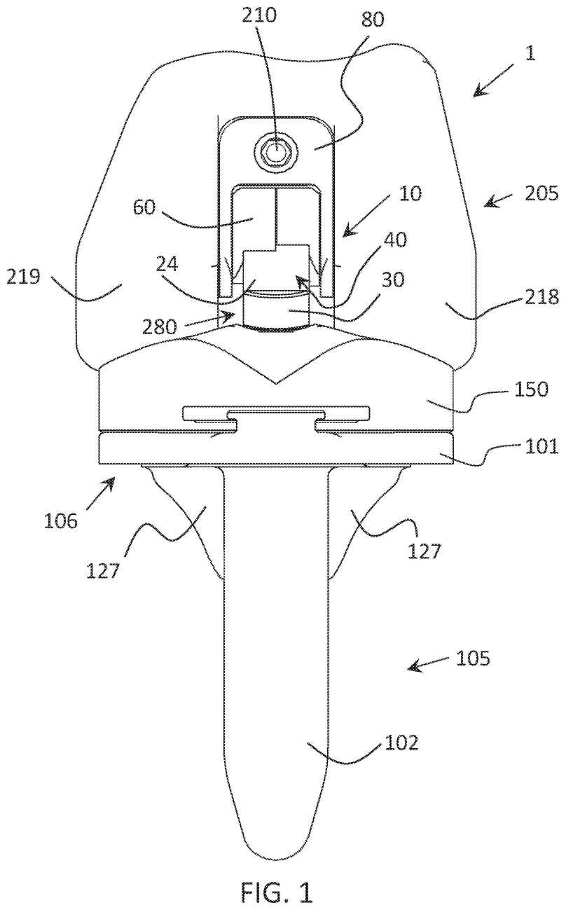

As shown in the assembled front view of FIG. 1, a rotating hinge knee endoprosthetic implant assembly 1 in accordance with this disclosure includes generally: a femoral component 205, a tibial component 105, a meniscal insert 150 disposed between the femoral component 205 and the tibial component 105, and a rotating hinge subassembly 10. In FIG. 1, many of the rotating hinge subassembly components are obstructed by the femoral component 205 and the tibial component 105, but some components are visible, such as a femur box 80 disposed in the femur box cavity 280 (FIG. 2A) of the femoral component 205, and a head 24 of the tibial yoke 40. Bearings 60 are disposed between the head 24 and the femur box 80 to facilitate the hinge movement of the rotating hinge knee endoprosthetic implant assembly 1. A femur fastener 210 extends through a femur box securing bore 81 in the femur box 80 and a femur securing bore 87 (FIG. 2A) in the femoral component 205 to fixedly engage the rotating hinge subassembly 10 to the femoral component 205. In the depicted embodiment, the femur fastener 210 is a tapered screw and the femur box securing bore 81 and the femur securing bore 87 are threaded to engage the corresponding threads of the tapered screw. In other exemplary embodiments, either the femur securing bore 87, the femur box securing bore 81, or both the femur securing bore 87 and the femur box securing bore 81 need not be threaded. An extension stop 30 can be provided to prevent overextension of the rotating hinge knee endoprosthetic implant assembly 1. In certain exemplary embodiments, the extension stop 30 can be a modular extension stop 30. That is, a surgeon can select and install one of a variety of available extension stops based on the patient's specific anatomy.

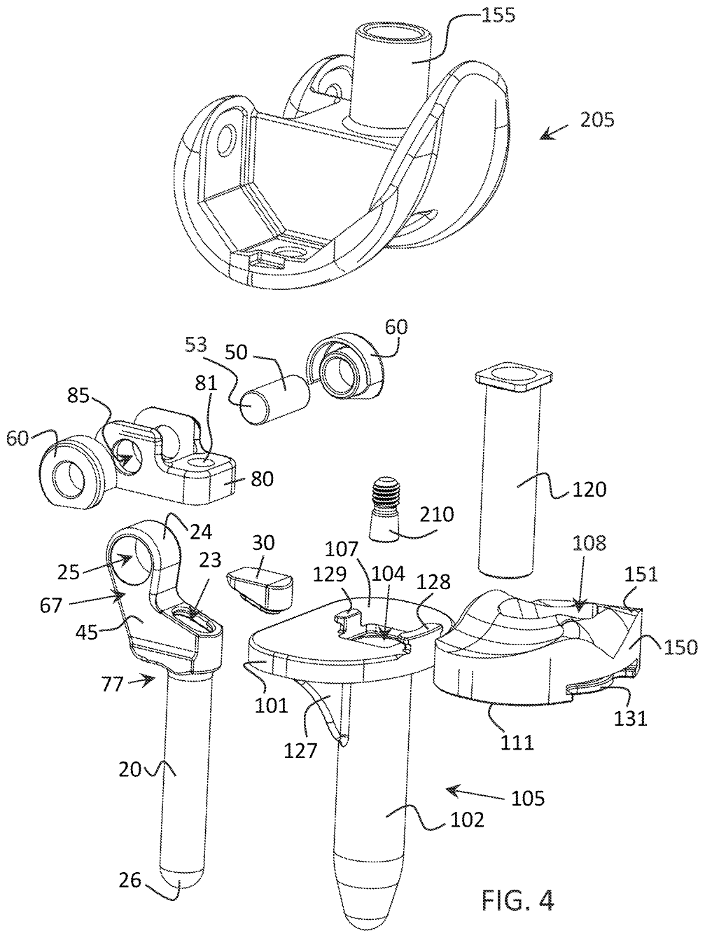

The femoral component 205 comprises a medial implant condyle 218 that is distally disposed from a lateral implant condyle 219. FIG. 1 depicts the respective implant condyles 218, 219 resting upon an articular surface 151 (FIG. 2A) of a meniscal insert 150. The meniscal insert 150 is typically made from medical grade polyethylene (e.g., ultra-high molecular weight polyethylene (“UHMWPE”)) or other suitable clinically tested biocompatible material. A base portion 101 of the tibial component 105 supports the meniscal insert 150. The base portion 101 is configured to rest on a patient's resected tibial plateau (103, FIG. 2C). The tibial component 105 can be made from biocompatible material, such as a cobalt-chromium molybdenum alloy, titanium alloy, or other clinically proven high-strength biocompatible material. A tibial stem 102 depends downwardly from the distal side 106 of the tibial base portion 101. The tibial stem 102 is configured to be inserted into an intramedullary bore (see the depicted cutaway 109) of the tibia 100. Keels 127 facilitate installation and securing of the tibial component 105 into an intramedullary bore (see 109) in the tibia 100 (see FIG. 2C). The depicted cutaway 109 is provided to illustrate how the tibial component 105 can be seated in and on the proximal tibia 100 when installed. In practice, the depicted cutaway 109 should generally not exist. It will be appreciated that other tibial components 105 that are compatible with the exemplary assemblies described herein may lack keels 127. In certain exemplary embodiments, the tibial stem 102 can be configured modularly to have extensions of different lengths to accommodate intramedullary bores of different lengths, angles, or offsets relative to the transverse plane 198 (FIG. 2D). In still further exemplary embodiments, the keels 127 can be modular components configured to fixedly engage the tibial component 105. As better seen in FIG. 4, the tibial stem 102 is substantially hollow on at least an upper/proximal end thereof and has an axial bore 104 configured for the receipt of a longitudinal tibial axial post 20 of a rotating hinge subassembly 10, as further described below.

Desirably, a sleeve 120 is inserted into the axial bore 104 prior to insertion of the tibial axial post 20. The sleeve 120 prevents the tibial axial post 20, which is typically made from a biocompatible metal alloy, from rubbing against the inside of the tibial stem 102, which is also typically made from a biocompatible metal alloy. Without the sleeve 120, it is contemplated that the repeated friction of the tibial axial post 20 moving against the inside of the tibial stem 102 could create metal shavings that could undermine the effectiveness and integrity of the endoprosthesis. It is contemplated that the sleeve 120 can be made from UHMWPE, polyether ether ketone (“PEEK”), or other clinically proven biocompatible polymer. In other exemplary embodiments, the sleeve 120 can be made from ceramic materials, including but not limited to zirconia toughened alumina (“ZTA”) ceramics. It still other exemplary embodiments, the sleeve 120 can be manufactured from cobalt chromium molybdenum alloys, or titanium allows and coated in zirconium oxide or niobium nitride to further reduce coefficients of friction between the articulating components and to enhance durability. In this manner, the sleeve 120 effectively creates a barrier between the inner wall of the tibial stem 102 and the outer wall of the tibial axial post 20.

FIGS. 2A-2D provide a series of perspective views showing primary steps of the implantation of the rotating hinge knee endoprosthetic implant assembly 1.

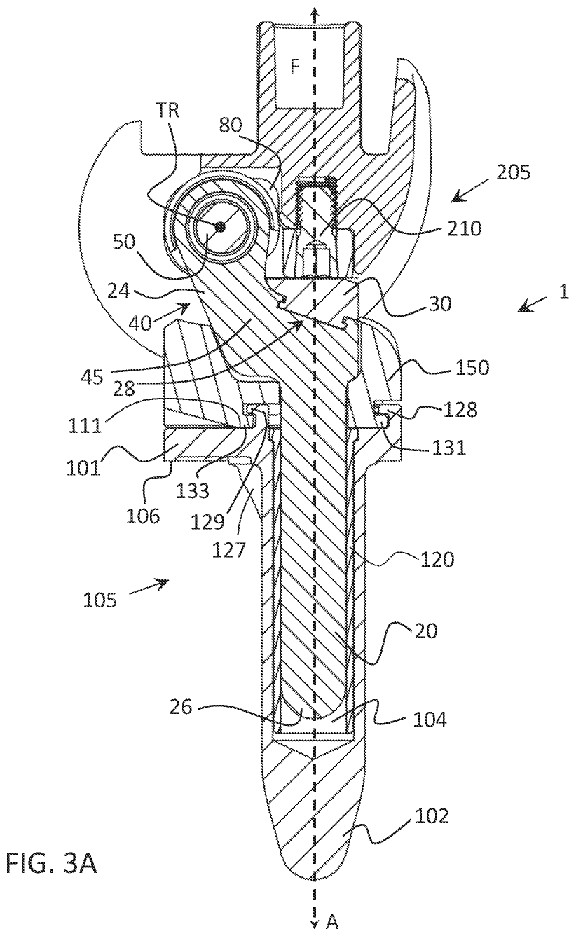

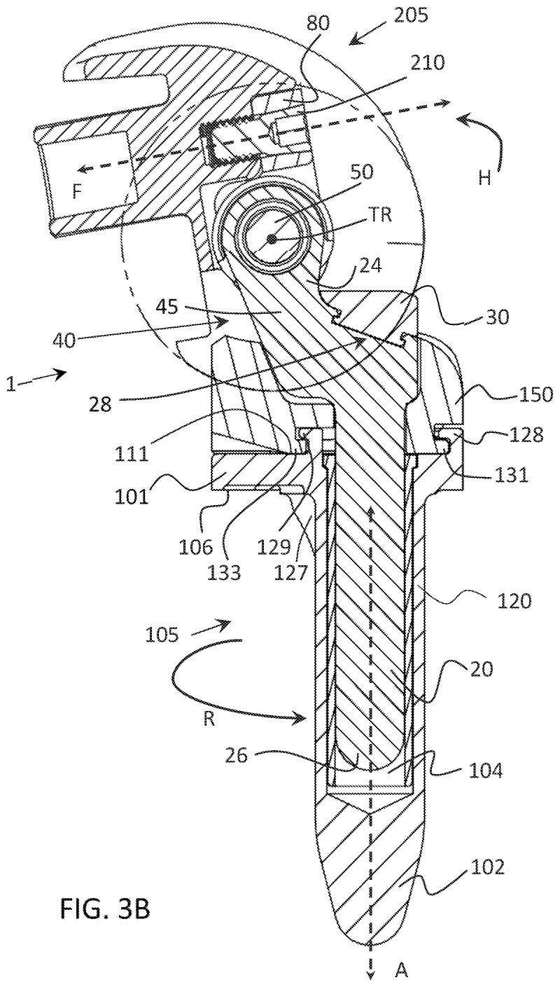

FIG. 2A depicts the femoral component 205 oriented in flexion relative to the tibial component 105. A large femur box cavity 280 is disposed between the medial implant condyle 218 and the adjacent lateral implant condyle 219. The femur box cavity 280 is configured to receive a femur box 80, as described below. In the depicted embodiment, the femoral component 205 comprises a femur securing bore 87 that is exposed to the femur box cavity 280. The femur securing bore 87 is secured in a transverse surface 92 that extends between the inner surfaces of the medial implant condyle 218 and the lateral implant condyle 219. A meniscal insert 150 is disposed on the base 101 of the tibial component 105 in a rotating or mobile bearing arrangement (see FIG. 3A for a discussion of an exemplary rotating bearing arrangement). Various configurations can be used for the connection between the meniscal insert 150 and the tibial base portion 101.

The femoral component 205 can be made from biocompatible material, such as a cobalt-chromium molybdenum alloy, titanium alloy, or other suitable high-strength biocompatible material. The articular surfaces (i.e., medial implant condyle 218 and a lateral implant condyle 219) can optimally be coated with a durable biocompatible material that has a low coefficient of friction to provide smooth articular bearing surfaces. Examples of such a coating include zirconium oxide or niobium nitride.

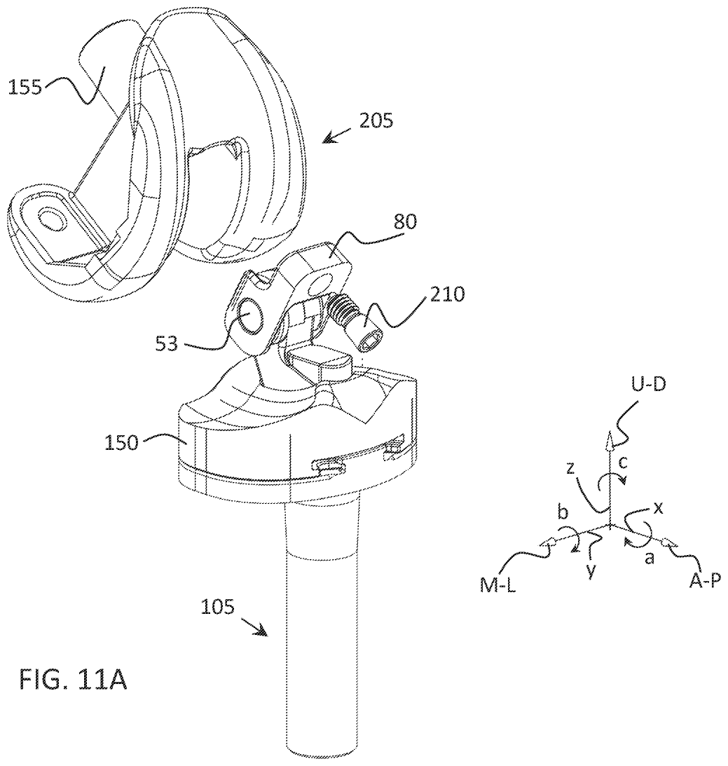

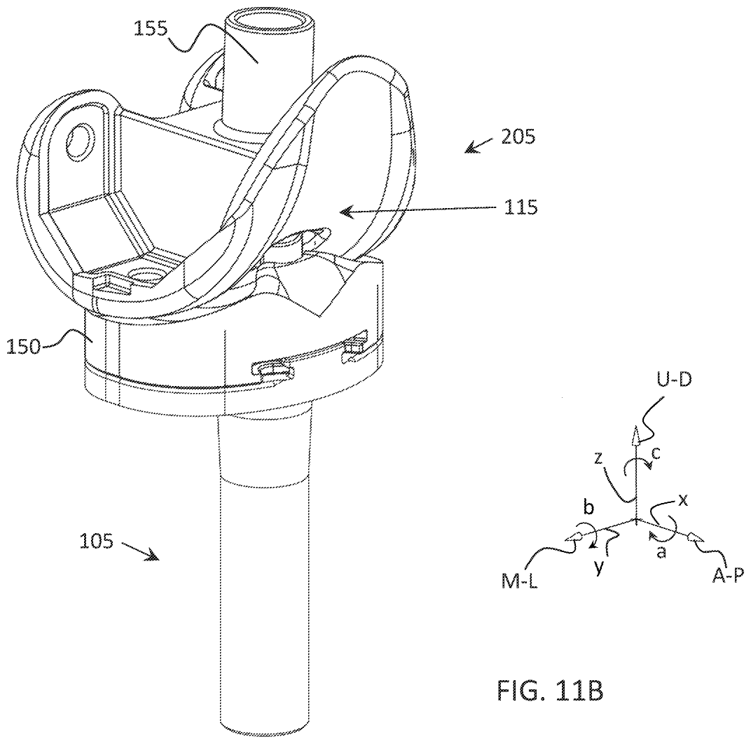

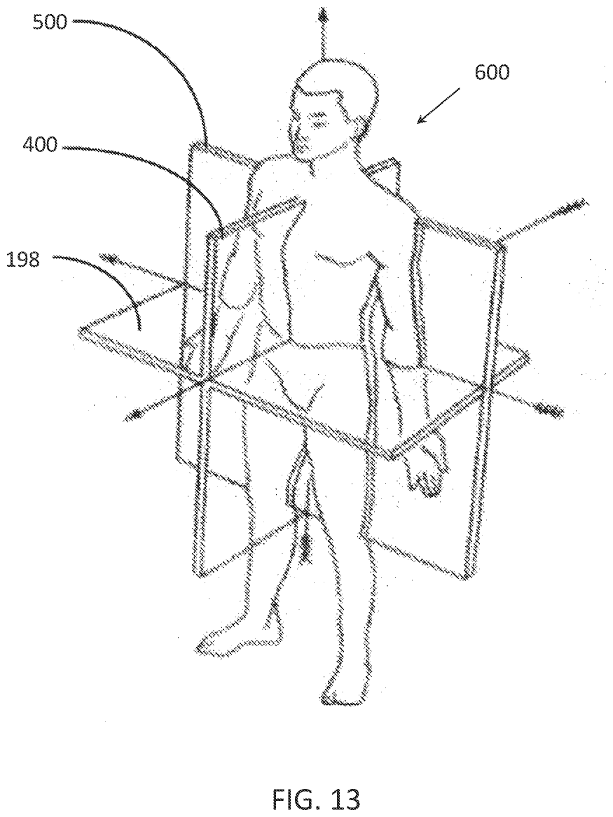

FIG. 2B provides a perspective view of a rotating hinge subassembly 10 in a disengaged position, wherein the first joint element (e.g., the femur box 80) is fully separated from the femoral component 205. One of the advantages of exemplary embodiments of the present disclosure is that the rotating hinge subassembly 10 can be completely assembled prior to insertion into the patient's knee, thus facilitating the implantation procedure and contributing to reduced operating time. A preassembled rotating hinge subassembly 10 comprises a yoke 40 and a femur box 80 that hingedly articulates around the head 24 of the yoke 40. The depicted femur box 80 comprises a femur box securing bore 81. It will be appreciated that a femur box securing bore 81 is an example of a femoral fastening mechanism that can be used to selectively engage the femur box 80 to the femur box cavity 280 of the femoral component. In other exemplary embodiments, it will be appreciated that the femoral fastening mechanism may comprise a projection, a recession, a receiver, multiple projections, multiple recessions, multiple receivers, part of a projection-receiver locking mechanism, a magnet, a clamp, a hook, a lip, a welding agent, a bonding agent, an adhesive, or combinations thereof. In other exemplary embodiments, the femoral fastening mechanism can comprise a pin inserted through the femur box securing bore 81 and the femur securing bore 87. In such exemplary embodiments, a surgeon may use a small hammer to deform a distal end of the inserted pin in the femur securing section to thereby fixedly engage the femur fastening mechanism to the femoral component in a parasagittal plane z (FIG. 11A, see also FIG. 13). In such embodiments, the femur securing bore 87 may have a maximum diameter that is greater than the maximum diameter of the femur box securing bore 81. The increased volume of the femur securing bore 87 relative to the femur box securing bore 81 may allow the distal end of the pin to deform in the femur securing bore 87 to thereby lock the pin femur fastener in a projection-receiver locking manner. Without being bound by theory, it is contemplated that the elimination of threaded elements in the femur fastener 210 may further mitigate the possibility of premature failure resulting from any minor incidence of torsional force transfer into the femur fastener 210.

In still other exemplary embodiments, the femoral fastening mechanism may comprise magnetic elements of opposite polarity in which a first magnetic element is disposed in the femur box 80 and the second magnetic element of opposite polarity from the first magnetic element is disposed in a transverse surface 92 that extends between the inner surfaces of the medial implant condyle 218 and the lateral implant condyle 219 of the femoral component 205.

An exemplary rotating hinge subassembly 10 may further comprise a modular extension stop 30 that can be configured to snap fit into an extension stop portion 28 (FIG. 3A) of the yoke 40. One or more bearing members 60 are pivotally connected to the head 24 of the yoke 40 (for example) via a transverse hinge pin 50 (FIG. 4). An end 53 of the transverse hinge pin 50 is visible in FIG. 2B. A lower or inferior portion of the body 45 of the yoke 40 includes a tibial axial post 20 configured to be disposed in the tibial component 105 in a rotating relationship. The tibial axial post 20 comprises a distal end 26. Details about these components are provided herein. It will be appreciated that in other exemplary embodiments, one bearing member 60 can be used. In still other exemplary embodiments, more than two bearing members 60 can be used.

FIG. 2C depicts the femoral component 205 having been secured to the distal femur 200 and the tibial component 105 having been secured to the proximal tibia 100 of the patient's knee joint using the resection and implantation techniques described above. FIG. 2C shows the rotating hinge subassembly 10 just prior to insertion and attachment to the implant assembly (i.e., the femoral component 205, the meniscal insert 150, and the tibial component 105). As indicated, the tibial axial post 20 is inserted into the axial bore (104, FIGS. 3A, 4) in the tibial stem 102. The femur box cavity 280 is sized and configured to allow the tibial axial post 20 to be inserted from a generally anterior or superior-to-inferior orientation when the femur 200 is in flexion.

FIG. 2D shows the rotating hinge subassembly 10 being attached to the hinge femoral component by a femur fastener 210. As can be appreciated in FIG. 2D, with the knee in flexion, the tibial axial post 20 of the yoke 40 (see also, FIG. 4) has been inserted into the axial bore 104 of the tibial stem 102 in a rotating relationship R and the femur box 80 has been seated in the femur box cavity 280. The femur fastener 210 is inserted through a securing bore 81 in the femur box 80 and through a femur securing bore 87 (i.e., a second joint element) in the femoral component 205 and in this manner secures the femur box 80 (i.e., the first joint element) to the femoral component 205. Once the femur fastener 210 secures the femur box 80 to the femoral component 205, the first joint element is in the engaged position. That is, the first joint element (e.g., the femur box 80) is disposed in the femoral component 205 and engages the femoral component 205 in a projection-receiver locking manner.

In this manner, the rotating hinge subassembly 10 is secured to the femoral component 205 and can be said to “fixedly engage” the femoral component 205. Other methods of selectively mechanically engaging a first joint element of the rotating hinge subassembly 10 to the femoral component 205 in a projection-receiver locking manner, a magnetic locking manner, a clamping locking manner, a bonding locking manner, an adhesive locking manner, or combinations thereof can also be said to “fixedly engage” the rotating hinge subassembly 10 to the femoral component 205.

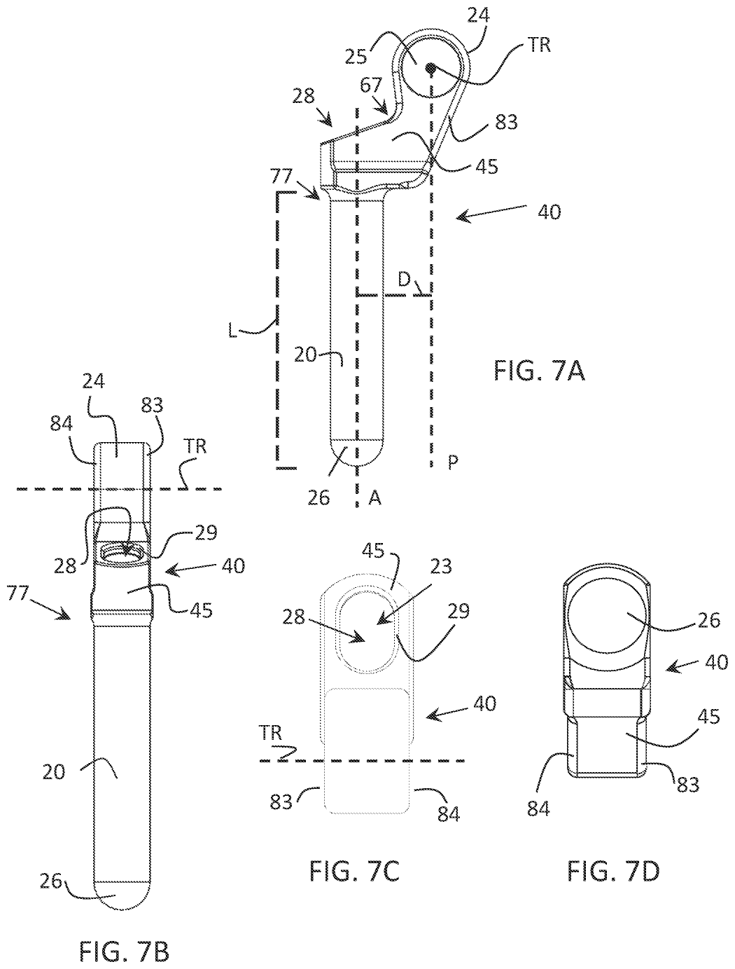

The length of the tibial axial post 20 (see FIG. 3A), the surrounding unresected soft tissue of the knee (e.g., the medial collateral ligament “MCL” and the lateral collateral ligament “LCL”), and the position of the femoral component 205 relative to the tibial component 105 effectively secures the tibial axial post 20 in the tibial component 105 while permitting the tibial axial post 20 to rotate around a tibial axis of rotation A during use. In this manner, the rotating hinge subassembly 10 can be said to be disposed in a hinged and rotating configuration in the rotating hinge knee endoprosthetic implant assembly 1. That is, the rotating hinge knee endoprosthetic implant assembly 1 is now capable of pivoting around the hinging component (i.e., “hingedly articulating,” see also the hinge direction of rotation H around the transverse hinge pin 50 in FIG. 3B), thereby permitting the rotating hinge knee endoprosthetic implant assembly 1 to flex and extend as is readily apparent in normal use, and the femur 200 is also now capable of rotating slightly axially around a generally vertical tibial axis of rotation A as the rotating hinge knee endoprosthetic implant assembly 1 undergoes flexion and extension. The length L (FIG. 7A) of the tibial axial post 20 and the surrounding soft tissue prevents the yoke 40 from dislocating from the axial bore 104 of the tibial stem 102 when the knee is in full flexion. However, the distance between this distal end 26 of the tibial axial post 20 and the bottom of the tibial axial stem 102 may increase when the knee is in full flexion.

This axial rotation R of the femur 200 relative to the tibia 100 approximates the natural movement of a knee joint. As such the rotating hinge knee endoprosthetic implant assembly 1 avoids some of the wear forces that a fixed hinge knee would be expected to experience during normal use. Because the rotating hinge knee endoprosthetic implant assembly 1 better approximates the natural movement of a normal knee compared to a fixed hinge design, rotating hinge knees can improve patients' post-operative comfort.

In other exemplary embodiments, the tibial axial post 20 may be fixedly engaged to the inside of the tibial stem 102, or a sleeve 120 (FIG. 4) so as to inhibit dislocation of the tibial axial post 20 from the axial bore 104 of the tibial stem 102. In such embodiments, the tibial axial post 20 may comprise an expandable element that can selectively extend radially away from the tibial axial post 20 to engage the inside surface of the tibial stem 102 or the sleeve 120 (whichever is present). In still other exemplary embodiments, tibial axial post 20, the inside of the tibial stem 102, or the inside and outside of the sleeve 120 may have protrusions that abut an adjacent element when the tibial axial post 20 is installed inside the tibial stem 102 to fixedly engage the post member therein. In yet other exemplary embodiments, the tibial axial post 20 and the adjacent structure (e.g., the sleeve 120 or the inside of the tibial stem 102) may comprise magnets of opposite polarity.

Without being bound by theory, it is contemplated that such embodiments may be desirable in patients that suffer from ligamentous laxity. Normally, the length L (FIG. 7A) of the installed tibial axial post 20 coupled with tension from relatively nominal surrounding soft tissue (including the MCL and LCL) prevents the tibial axial post 20 from dislocating from the axial bore 104 of the tibial stem 102 when the knee is in full flexion. However, in patients that suffer from ligamentous laxity, the MCL and LCL (and other surrounding soft tissue) may not exhibit sufficient tension to prevent the dislocation of the depicted tibial axial post 20 from a depicted sleeve 120, or inside of the tibial stem 102. As such, fixedly engaging the tibial axial post 20 to the inside of the tibial stem 102, or a sleeve 120 in the manner described may be desirable to prevent dislocation.

Referring back to FIG. 2D, the depicted femur fastener 210 is a tapered screw. The depicted tapered screw is generally shorter than fasteners used in other rotating hinge knees. Moreover, the tapered screw having threads disclosed in the exemplary embodiment is secured to the femoral component 205, whereas some other rotating hinge assemblies are secured with threads to the tibial component.

Without being bound by theory, it is contemplated that the significant compressive and torsional loads that the tibia 100 and tibial component 105 experience during normal use of the knee can contribute to loosening of such tibial fastening elements and can eventually compromise the future stability and effectiveness of the endoprosthesis. In designs where the tibial fastening screw is locked, normal compressive and torsional forces can eventually wear away the screw threads, thereby also loosening the rotating hinge component and compromising the effectiveness of the implant.

In embodiments in accordance with this disclosure, the condyles 218, 219 desirably transfer a majority of the femoral load (the femoral load comprising the compressive load of the body above and including the femur and torsional loads) to the condylar pads 228, 229 of the meniscal insert 150. This is typically known as “condylar loading.” The meniscal insert 150 then transfers the femoral load through the tibial component 105, tibia 100 and ultimately the patient's foot during normal use. In this manner, the disclosed embodiments desirably avoid transferring excessive forces from the femur into the rotating hinge subassembly 10.

However, in practice, it is contemplated that not all of the femoral loads will be transferred to the condylar pads 228, 229 of the meniscal insert 150. Some of the compressive and torsional loads, as well as other loads such as varus, valgus, hyper-extension, flexion, and anterior and posterior drawer forces can be transferred from the femur 200 through the rotating hinge subassembly 10. In such cases, it is contemplated that the femoral loads are transferred from the femur 200 and femoral component 205 to the femur box 80 (i.e., the first joint element) and through the transverse hinge pin 50 (FIG. 3A) into the head 24 and body 45 of the yoke 40 (FIG. 7A). The portions of the yoke body 45 that are disposed within the meniscal insert 150, then transfer these forces into the tibial component 105, tibia 100 and ultimately the patient's foot during normal use. Without being bound by theory, it is contemplated that even in situations in which forces are transferred into the rotating hinge subassembly 10, the transverse hinge pin 50 and yoke 40 transfer these forces through the remainder of the leg while avoiding transferring these forces to the femur fastener 210. That is, the femoral load is transferred through the femur box 80 and possibly the transverse hinge pin 50 rather than the femur fastener 210 as described further below. By placing the femur fastener 210 in a location that permits the femur fastener 210 to mechanically engage the first joint element (e.g., the femur box 80) to the femoral component 205 while removing the femur fastener 210 from the chain of force transfer, it is contemplated that embodiments in accordance with the present disclosure can avoid fastener cross threading or other signs of premature wear that would otherwise result from normal use. In certain exemplary embodiments, the location that the femur fastener 210 or other femoral fastening mechanism that mechanically engages the femur box 80 to the femoral component 205 may desirably be at a location that is co-axial with tibial rotation axis A when the knee is in extension (see FIG. 3A). Without being bound by theory, it is contemplated that having the femoral component 205 being coaxial with the tibial rotation axis A can further minimize torsional and other ancillary loads on the femur fastener 210 or other femoral fastening mechanism.

FIG. 3A provides a lateral cross-sectional extension view in which further aspects of the interconnection between the components can be visualized. The cross-section is taken along an implant-bisecting parasagittal plane (see 400, FIG. 13). The knee is shown in full extension (i.e., at zero degrees of flexion). In the depicted embodiment, the tibial axis of rotation A is substantially aligned (i.e., is collinear) with the femur fastener's central axis F when the rotating hinge knee endoprosthetic implant assembly 1 is in extension, but not when the rotating hinge knee endoprosthetic implant assembly 1 is in flexion (see the femur fastener's central axis F relative to the tibial axis of rotation A in FIG. 3B). The vector of the femur fastener's central axis F moving toward the femoral component 205 can be representative of the engagement direction. The vector of the femur fastener's central axis F moving away from the femoral component 205 can be representative of a disengagement direction.

A modular extension stop 30 is provided. As discussed in more detail below, the modular extension stop 30 can be configured to snap fit into an extension stop portion 28 of the yoke 40. Extension stops are generally used to prevent recurvatum. By making the extension stop 30 modular, it is contemplated that the surgeon can select a modular extension stop 30 that best fits the patient's need. Additionally, in some embodiments the modular extension stop 30 is omitted and an extension stop that is secured to the rotating hinge subassembly 10 in a fixed configuration can instead be provided. In such cases, the degree of potential hyperextension is limited by the patient's anatomy and by the interaction between the condyles 218, 219 of the femoral component 205 and the raised anterior surface of the meniscal insert 150.

As shown in FIG. 3A, the femur box 80 is secured to the femoral component 205 by the femur fastener 210 extending through the femur box securing bore 81. Further, the femur box 80 and a bearing member 60 are hinged to the head 24 of yoke 40 by the transverse hinge pin 50. In this manner, the femur 200 rotates (see H, FIG. 3B) about a single transverse axis TR relative to the head 24 of the yoke 40. With the tibial axial post 20 resting in the axial bore 104 of the tibial stem 102, the femur 200 is also free to rotate relative to the tibial component 105 about the generally midline tibial axis of rotation A. The degree of rotation is restricted by the patient's anatomy and by the interaction between meniscal insert 150 and the tibial component 102, which is described further below. In the embodiment of FIG. 3A, the tibial axial post 20 passes through a through-bore 108 (FIG. 4) formed in the meniscal insert 150 and into the axial bore 104 of the tibial stem 102. In this manner, the rotating hinge subassembly 10 in the engaged position secures the meniscal insert 150 to the rotating hinge knee endoprosthetic implant assembly 1. In the embodiment of FIG. 3A, the modular extension stop 30 is configured to snap-fit onto the extension stop portion 28 in the body 45 of the yoke 40 in a fixed arrangement.

FIG. 3A further depicts the base portion 101 of the tibial component 105 further comprising an anterior hook 128 and a mid-hook 129. Each hook 128, 129 defines a negative space between the bottom of the hooks 128, 129 and the surface of the tibial base portion 107. The meniscal insert 150 has an anterior protrusion 131 and a mid-protrusion 133 disposed at the base 111 of the meniscal insert. The anterior protrusion 131 and a mid-protrusion 133 desirably fill the negative space defined by the bottom of the anterior hook 128 and a mid-hook 129 when the meniscal insert 150 is installed on the tibial base portion 101. In this manner, the meniscal insert 150 is not only configured to snap-fit onto the tibial base portion, but the arrangement of the hooks 128, 129 and the protrusions 131, 133 permits and also limits rotational sliding of the meniscal insert 150 around the generally midline tibial rotational axis A during normal flexion and extension of the rotating hinge knee endoprosthetic implant assembly 1.

It will be appreciated that if the surgeon elects not to dislocate the entire length of the tibial axial post 20 from the tibial stem 102, the presence of the hinge subassembly 10 in the engaged position prevents the meniscal insert 150 from sliding out of the tibial component 105 when the knee is lifted in flexion during ambulatory movement. Other manners of securing the meniscal insert 150 to the tibial base 101 such that the meniscal insert 150 is rotatable around the generally midline axis of the tibia A are considered to be within the scope of this disclosure. A sleeve 120 can optionally, but desirably be disposed in the tibial stem 102 between the tibial axial post 20 and the tibial stem 102. The sleeve 120 can be tightly fitted to the internal diameter of the tibial stem 102 and the outer diameter of the tibial axial post 20.

FIG. 3B provides a side cross-section deep flexion view in which further aspects of the interconnection between the components can be visualized. The rotating hinge subassembly 10 can be configured to provide deep flexion in the range of about 100 degrees to about 138 degrees, which is ideal for a revision knee. In certain exemplary embodiments, the range may be between about 100 degrees and about 125 degrees. In deep flexion, maximum rotation of the tibial axial post 20 relative to the tibial component 105 is achieved, but is also limited by the hooks 128, 129 and the protrusions 131, 133 of the meniscal insert 150 and tibial component 102. In addition to the pivoting motion, the femur 200 continues to hinge around the tibial axial post 20 only along the axis of the transverse hinge pin 50.

As indicated in the side cross-sectional views of FIGS. 3A and 3B, the degree of translation of the tibial axial post 20 in the axial bore 104 during flexion is determined by the configuration of the condyles 218, 219 of the femoral component 205. If the condyles 218, 219 have a single fixed radius, the tibial axial post 20 will not translate to an appreciable degree during flexion. Likewise, if the condyles 218, 219 have a two or more radii, the tibial axial post 20 will translate upwardly in the axial bore 104 during flexion.

Aspects and features of the individual components of the rotating hinge knee endoprosthetic implant assembly 1 will now be discussed.

FIG. 4 provides an exploded view of one exemplary embodiment of a rotating hinge knee endoprosthetic implant assembly 1. Most of these components have been discussed above. However, additional components can be seen in the exploded view that were not clear or visible in the prior images. Reference is made to the other figures for a detailed overview of the components.

Femur Box

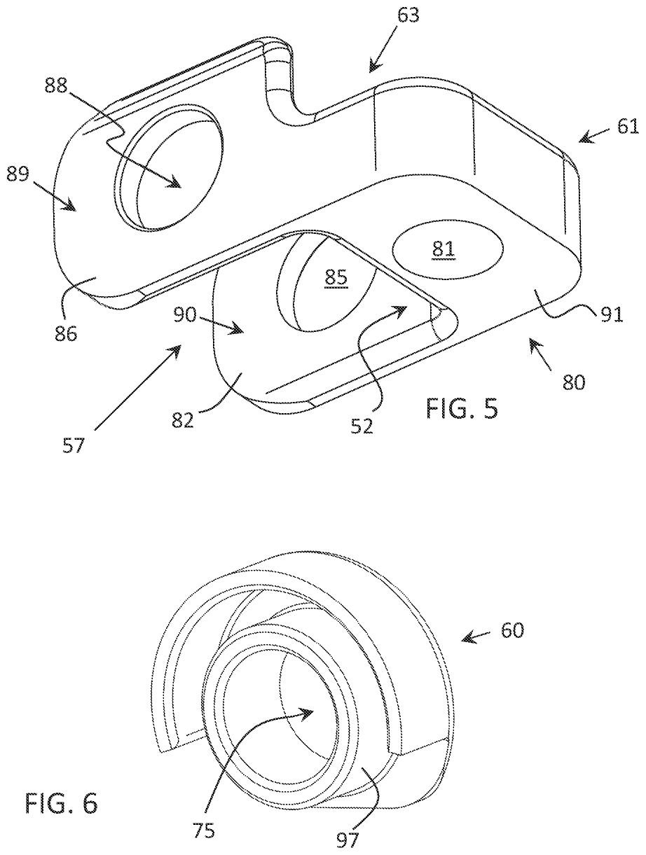

FIG. 5 shows a perspective view of one embodiment of a femur box 80 (i.e., an exemplary first joint element) for use in a rotating hinge knee endoprosthetic implant assembly 1 of the present disclosure. In the embodiment of FIG. 5, the femur box 80 has a generally clevis configuration. The femur box 80 includes a main body portion 91 on anterior end 61 and a pair of opposing arms 82, 86 extending posteriorly therefrom toward a posterior end 52. In the depicted embodiment, the arms 82, 86 take the form of clevis arms. The pair of opposing arms 82, 86 define a gap 57 between the inner surfaces 90 of the opposing arms 82, 86. The gap 57 is sized to accommodate the width of the head 24 of the yoke 40 and desirably the width of any attached bearing members 60. The main body portion 91 of the femur box 80 is provided with a femur box securing bore 81 extending therethrough. Additionally, the opposing arms 82, 86 are each respectively provided with arm bore 85, 88 (i.e., the first arm 82 defines a first arm bore 85, the second arm 86 defines a second arm bore 88) extending therethrough for receiving the transverse hinge pin 50 (FIG. 4). The arm bores 85, 88 are formed in the respective arms 82, 86. The pair of arm bores 85, 88 are substantially axial in alignment with one another (along the transverse axis TR) to thereby provide a continuous hinge pin bore spanning the opening between the arms 82, 86.

In certain exemplary embodiments, the femur box 80 can be made from a durable clinically proven biocompatible material that can support repeated force transfers from the femur fastener mechanism over the life of the endoprosthetic implant 1. Example materials include cobalt chrome molybdenum alloys and titanium alloys. In other exemplary embodiments, the femur box 80 can be made from polyether ether ketone “PEEK,” an organic thermoplastic polymer. PEEK is hydrophobic, which reduces any risk of the thermoplastic fusing with bone. Such properties may contribute to reduced wear of the PEEK component over time. PEEK is also radiolucent and non-magnetic, thereby making PEEK components transparent on radiographs and compatible with magnetic imaging technologies.

In other exemplary embodiments, it is contemplated that and the femur box 80 can be made from other biocompatible, clinically proven articulating materials, including but not limited to UHMWPE and ceramic materials, including but not limited to zirconia toughened alumina (“ZTA”) ceramics. It is further contemplated that if the femur box 80 is manufactured from metal, the femur box 80 may be optionally coated in zirconium oxide or niobium nitride to further reduce coefficients of friction between the articulating components and to enhance durability. In such exemplary embodiments, it is contemplated that coating the outer surface of the femur box 80, including the outside faces of the arms 82, 86 may be desirably coated further reduce coefficients of friction. For clarity, the femur wall defining the box securing bore 81 should not be coated with a friction-reducing substance because such a substance would facilitate removal of the femur fastener 210 during normal use.

As indicated in the drawings, the femur box 80 is sized and configured to house all components of the hinge function. In embodiments, the transverse hinge pin 50 is sized to fit flush on the outside faces of the arms 82, 86. In certain exemplary embodiments, the edges 63 of the femur box 80 are radiused for unobstructed assembly into the femur implant.

Without being bound by theory, it is contemplated that sizing the transverse hinge pin 50 to have the ends 53 of the transverse hinge pin 50, that are disposed flush or nearly flush to the outside faces 89 of the arms 82, 86, permits the femoral component 205 to transfer any torsional forces from the femur 200 to the femur box 80 over the combined surface areas of the outside face 89 of the arms 82, 86 and the respective ends 53 of the transverse hinge pin 50. It will be understood that “flush” or “flushly” as used in this context means that the ends 53 of the transverse hinge pin 50 are disposed substantially coplanar with the plane that is coextensive with the outside face 89 of one or both of the arms 82, 86.

It is contemplated that transferring any torsional forces in this manner can distribute the torsional load over a larger area thereby reducing the concentration of the torsional load in any one area. It is further contemplated that the distribution of any torsional load over a broader area can reduce torsional wear and generally prolong the useful life of the exemplary embodiments described herein. In other exemplary embodiments, one or both ends 53 of the transverse hinge pin 50 may not be disposed flush with the outside face 89 of the arms 82, 86; rather, one or both ends 53 of the transverse hinge pin 50 may extend into the pair of arm bores 85, 88 to permit the femur box 80 to rotate around the transverse axis TR of the transverse hinge pin 50, but remain within the pair of arm bores 85, 88 such that one or both ends 53 of the transverse hinge pin 50 do not extend further than the outside face 89 of the arms 82, 86. It is contemplated that such embodiments may still provide the advantage of distributing torsional forces over a larger surface area than what was previously known.

Although the transverse hinge pin 50 is depicted as a separate element throughout the figures (see e.g., FIG. 4) it will be understood that in other exemplary embodiments, the inner sides 90 of the arms 82, 86 may comprise a portion of the transverse hinge pin 50. In such exemplary embodiments, a medial end of the portion of the transverse hinge pin 50 extends away from the inner surface 90 of the first arm 82 into the gap 57 between the opposing arms 82, 86. In an assembled configuration, the portion of the transverse hinge pin 50 extends into the transverse hinge bore 25 of the head 24 of the yoke 40 to thereby hingedly engage the first arm 82 of the femur box 80 to the yoke 40. Similarly, such exemplary embodiments may further comprise a second portion of the transverse hinge pin 50 extending away from the inner surface (view obstructed in FIG. 5, but see 90) of the second arm 86 into the gap 57 between the opposing arms 82, 86. In an assembled configuration, the second portion of the transverse hinge pin 50 extends into the transverse hinge bore 25 of the head 24 of the yoke 40 to thereby hingedly engage the second arm 86 of the femur box 80 to the yoke 40. In such exemplary embodiments comprising this modified femur box 80, the femur box 80 can be said to “press fit” or “interference fit” into the transverse hinge bore 25 of the yoke 40. Furthermore, in such exemplary embodiments, the arm bores 85, 88 may be absent.

Bearing Member

FIG. 6 depicts an example bearing member 60 sized to have a bearing surface that extends into the transverse hinge bore 25 of the head 24 of the tibial yoke 40. This bearing member 60 can rotate freely within the femur box 80 and around the yoke head 24 and the transverse hinge pin 50 when disposed in an installed position (see FIG. 2). It will be appreciated that certain exemplary embodiments can comprise more than one bearing member 60. The bearing member 60 defines a bearing bore 75 configured to closely receive the transverse hinge pin 50 (or a portion of the transverse hinge pin 50). In this manner, the transverse hinge pin 50 is supported by and rotates within the bearing bore 75. The ends 53 of the transverse hinge pin 50 are disposed in the axially aligned arm bore 85, 88 of the femur box 80. In this manner, the femur box 80 is configured to hingedly rotate around the transverse axis TR of the rotating hinge subassembly 10. Other embodiments may use a bearing sleeve that circumferentially abuts the transverse hinge pin 50 and extends through the arm bores 85, 88 to achieve hinging rotation around the transverse rotational axis TR.

It will be understood that in other exemplary embodiments, the inner side of the bearing member 60 may comprise a portion of the transverse hinge pin 50. In such exemplary embodiments, a medial end of the portion of the transverse hinge pin 50 extends away from the inner surface of the bearing member 60. In an assembled configuration, the portion of the transverse hinge pin 50 extends into at least one arm bore 85 and into the transverse hinge bore 25 of the head 24 of the yoke 40 to thereby hingedly engage the bearing member 60 to the femur box 80 and the yoke 40. Multiple bearing members 60, each having a portion of the transverse hinge pin 50 extending from an inner surface are also contemplated. In such exemplary embodiments comprising this modified bearing member 60, the portion of the transverse hinge pin 50 can be said to “press fit” or “interference fit” into the bearing member 60.

In embodiments in which the bearing member 60 is configured to be disposed between a lateral side 84 of the yoke 40 and the inner surface 90 of an arm 86 of the femur box 80, the bearing member 60 can comprise an inner hinge portion oppositely disposed from an outer hinge portion, wherein the inner hinge portion and the outer hinge portion are coaxially aligned with the transverse rotation axis TR in an assembled configuration. The outer hinge portion is disposed within an arm bore 88 of an arm 86 of the femur box 80 while the inner hinge portion is disposed within the transverse hinge bore 25 of the yoke 40 to thereby hingedly engage the femur box 80 to the yoke 40. Such embodiments may further comprise a second bearing member 60 so described to hingedly engage the other arm 82 of the femur box 80 to the yoke 40. In all such exemplary embodiments, the end 53 of the transverse hinge pin 50 desirably does not extend into the femoral component 205 in the installed configuration.

In certain exemplary embodiments, it is contemplated that the bearing member 60 can be made from a biocompatible, clinically proven articulating materials, including but not limited to cobalt chrome molybdenum alloys, titanium alloys, UHMWPE, PEEK, and ceramic materials, including but not limited to zirconia toughened alumina (“ZTA”) ceramics. It is further contemplated that if the bearing member 60 is manufactured from metal, the bearing member 60 may be optionally coated in zirconium oxide or niobium nitride to further reduce coefficients of friction between the articulating components and to enhance durability. In such exemplary embodiments, it is contemplated that coating the outer surface of the bearing member 60, may be desirably coated further reduce coefficients of friction. The bearing effectively creates a barrier between the femur box 80 and the head 24 of the yoke 40. A zirconium oxide or niobium nitride coating may desirably substantially reduce the likelihood of metal shavings generated as the result of normal use that might otherwise arise with two metal components creating shear forces relative to one another during normal movement.

Tibial Yoke

FIG. 7A shows a lateral view of one embodiment of an endoprosthetic tibial yoke 40 (i.e., an exemplary second joint element) for use in a rotating hinge knee endoprosthetic implant assembly 1. In certain exemplary embodiments, the tibial yoke 40 is a unibody (i.e., a single contiguous) structure having a body member 45 and a tibial axial post 20 extending downwardly from the second end 77 of the body member 45. The second end 77 of the body member 45 is distally disposed from a first end 67 of the body member 45. The head 24 extends from the first end 67 of the body member 45. The head 24 defines a transverse hinge bore 25 extending therethrough. In other exemplary embodiments, the tibial axial post 20 and body member 45 or the body member and head 24 may be separately manufactured components that are desirably assembled into the tibial yoke 40 prior to insertion into the rotating hinge knee endoprosthetic implant assembly 1.

As seen in the lateral view of FIG. 7A, the body member 45 extends away from the transverse rotational axis TR to provide an offset distance D between the transverse rotational axis TR of the transverse hinge bore 25 and the tibial rotation axis A of the tibial axial post 20. In certain exemplary embodiments, the inner diameter of the transverse hinge bore 25 is sized to receive and articulate with an outer diameter of areas 97 of a bearing member 60 that define the bearing bore 75. The bearing bore 75 receives the transverse hinge pin 50, as described elsewhere herein. It will be appreciated that other ways of using a bearing member 60 to reduce the coefficient of friction between the femur box 80 and the transverse hinge pin 50 are withing the scope of this disclosure. In certain exemplary embodiments, the edges of the body member 45 are radiused.

The tibial axial post 20 further comprises a distal end 26 disposed distally from the second end 77 of the body member 45. The distal end 26 may be chamfered or otherwise configured to ease insertion of the tibial axial post 20 into the axial bore 104 of the tibial stem 102. In certain exemplary embodiments, the distal end 26 may comprise a conical tip. In other exemplary embodiments, the distal end 26 may be substantially hemispherical. In still other exemplary embodiments, the distal end 26 can be selected from a group of shapes including a generally convex shape, a chamfered shape, a convex conical shape, a convex hemispherical shape, and a convex frustoconical shape. In certain exemplary embodiments, the tibial axial post 20 can define a post member chamber 23, and thereby be hollow (see FIG. 7C).

FIG. 7D is a bottom-up view of an exemplary tibial yoke 40 depicting the distal end 26 of the tibial axial post 20, the body 45 of the yoke 40 and the first and second lateral sides 83, 84.

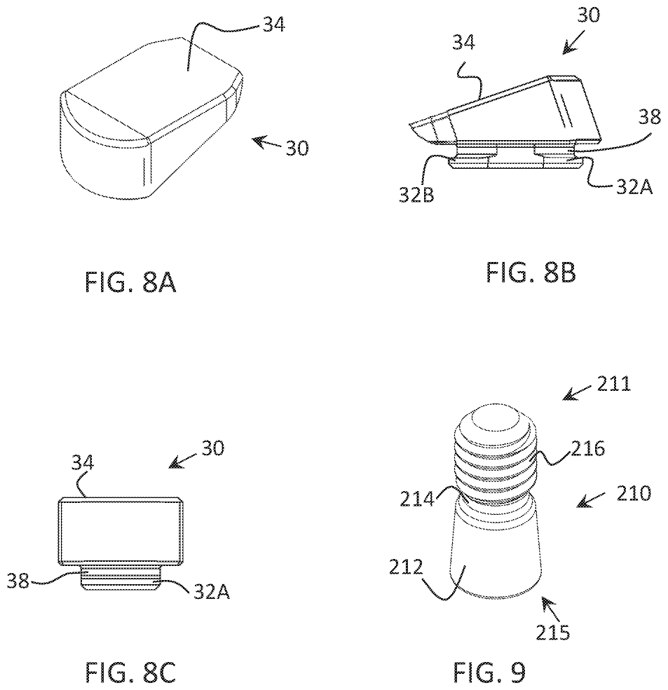

In certain exemplary embodiments, the body member 45 includes an extension stop portion 28 (FIG. 7B, 7C) extending on the top of the body member 45 between the head 24 and the tibial axial post 20. As indicated in FIG. 7A, in embodiments, an offset distance D between the midline tibial rotational axis A of the tibial axial post 20 and a parallel axis P extending orthogonally through the transverse axis TR of the transverse hinge bore 25 is about 12 mm to about 15 mm. The offset distance D defines the center of rotation of the femur 200 relative to the tibia 100. Certain exemplary embodiments may position revision femur's center of rotation slightly more anteriorly than the native femur's center of rotation. In these exemplary embodiments, it has been discovered that allowing the femur to rotate more anteriorly can contribute to better condylar loading and force transfer away from the femur fastening mechanism. In situations in which multiple yokes 40 are provided, the multiple yokes 40 may have different size dimensions, including different offset distances D. The surgeon may select the appropriately sized yoke 40 with the appropriately sized offset distance D based on the size and the integrity of a patient's particular anatomy. The extension stop portion 28 is configured to receive and connect to a separate modular extension stop 30, as described herein. The mechanism for connecting to the modular extension stop 30 can include snap-fit pockets 29 formed in an upper surface of the modular extension stop portion 28 for receiving matching locking tabs 32A, 32B (FIG. 8B) of the modular extension stop 30.

Modular Extension Stop

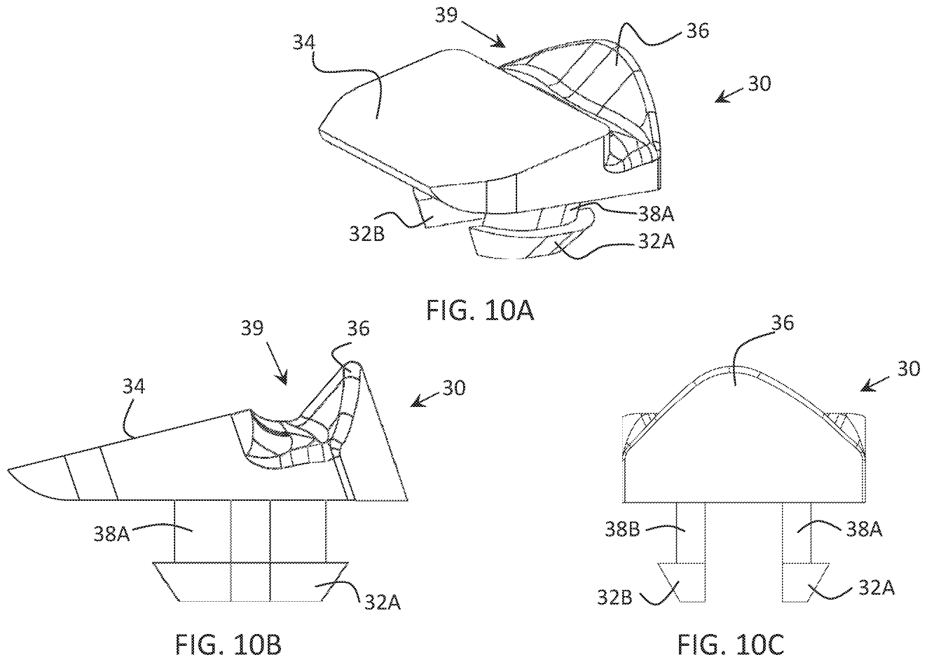

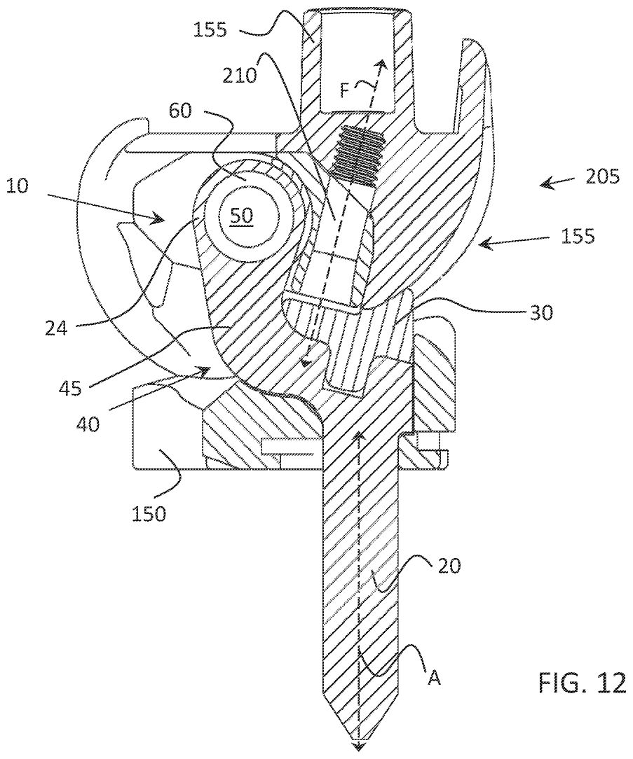

FIGS. 8A-8C show views of one embodiment of an extension stop 30 for use in an exemplary rotating hinge knee endoprosthetic implant assembly 1. The modular extension stop 30 can exist in an uninstalled position in which the modular extension stop 30 is not engaged to the rotating hinge subassembly 10. The modular extension stop 30 can also exist in an engaged position in which the modular extension stop is engaged to the extension stop portion 28 of the rotating hinge subassembly 10. In FIG. 8A, it can be seen that the modular extension stop 30 has a substantially flat or planar top surface 34 of a posterior side. In the embodiment depicted in FIG. 10, the top surface 34 rises into an abutment portion 36. The abutment portion 36 fits within the patellar groove of the femoral component 205 when the knee is in extension. In certain exemplary embodiments, the abutment portion 36 can have a concave anterior portion that continues the patellar groove of the femoral component 205 when the knee is in extension. In this manner, an exemplary modular extension stop 30 can be configured to fit within a patellar grove of the femoral component 205 and to continue the patellar groove defined by the femoral component 205, which permits a smooth transition between extension and flexion (see FIG. 12). A dip 39 separates the top surface 34 from the abutment portion 36. This dip 39 prevents the overextension (e.g., negative flexion, or bending of the knee in the wrong direction) of the femoral component 205.

In FIG. 10C, an anterior view of one embodiment of a modular extension stop 30 for use in an exemplary rotating hinge knee implant assembly of the present disclosure.

The modular extension stop 30 may be made of a durable plastic material such as UHMWPE. The modular extension stop 30 can be provided in different configurations to allow for various degrees of hyperextension, such as −10°, −5°, −3°, and 0° hyperextension. In this manner, the surgeon is able to readily customize the modular extension stop 30 to the individual patient.

In embodiments, the modular extension stop 30 is configured to snap lock into an extension stop portion 28 of the tibial axial post 20. In the embodiment shown in FIGS. 10A, 10B, and 10C, the snap locking mechanism can comprise a first snap member 32A disposed adjacently to a second snap member 32B. The snap members 32A, 32B can be bendable around respective snap member stems 38A, 38B. The snap members 32A, 32B can fit into snap fit pockets 29 of the extension stop portion 28 of the yoke 40 when the modular extension stop 30 is in an installed position. FIGS. 8A, 8B, and 8C depict alternative snap fit members 32A, 32B that are not bendable around a snap fit stem 38. Similar to the embodiments shown in embodiment shown in FIGS. 10A, 10B, and 10C the snap members 32A, 32B of FIGS. 8A, 8B, and 8C can fit into snap fit pockets 29 of the extension stop portion 28 of the yoke 40 when the modular extension stop 30 is in an installed position. All projection-receiver locking structures that can selectively mechanically engage the modular extension stop 30 to the extension stop portion 28 of the yoke 40 are considered to be within the scope of this disclosure.

In other exemplary embodiments, the extension stop 30 is not modular, rather the extension stop 30 is integrally connected to the yoke 40 at all times. In such embodiments the extension stop 30 can be manufactured as a part of the yoke 40, or the extension stop 30 can be permanently affixed to the extension stop portion 28 in a manner that precludes replacement of an extension stop 30 on the yoke 40 intraoperatively.

Tapered-Head Fastener

FIG. 9 shows a top down perspective view of one embodiment of a femur fastener 210 for use in a rotating hinge knee endoprosthetic implant assembly 1 of the present disclosure. In the pictured embodiment, the femur fastener 210 includes a threaded leading end 211, a tapered head 212 on a trailing end 215, and a smooth shank portion 214 between the threaded leading end 211 and the tapered head 212. The thread 216 is typically a machined thread. The threads 216 of a threaded portion approaching the threaded leading end 211 are desirably chamfered or tapered to facilitate the installation of the femur fastener 210 into the femur box securing bore 81. That is, the diameter of the femur fastener 210 at the threaded leading end 211 is desirably smaller than the inner diameter of the femur securing bore 87. Guiding a smaller diameter threaded leading end 211 into a comparatively larger diameter femur securing bore 87 permits the surgeon to be less precise when initially inserting the threaded leading end 211 of the femur fastener 210 into the femur securing bore 87 and thereby is configured to facilitate engagement of the femur box 80 to the femoral component 205 via the femur fastener 210.

To eliminate the possibility of cross-threading, surgeons can desirably start to insert the femur fastener 210 (if the femur fastener 210 has threads 216) by first rotating the femur fastener 210 in the opposite direction from the engagement direction. This allows for the leading thread of the femur fastener 210 to eventually drop below the leading edge of the threads in the femur securing bore 87. This may create an audible click, or the surgeon may simply register the sudden change in position. Once the surgeon registers the click or the engagement of the threads, the surgeon can then start to rotate the femur fastener 210 in the engagement direction.

As the femur fastener 210 threads into the femur securing bore 87 of the femoral component 205, the tapered head 212 automatically locks into the femur box securing bore 81 to prevent the femur fastener 210 from backing out of the femoral component 205. The tapered locking configuration of the femur fastener 210 enables distal assembly of the rotating hinge knee endoprosthetic implant assembly 1. “Distal assembly” means the assembly on the femoral component 205, which is secured to the distal portion of the patient's resected femur 200 by this point in the installation procedure. It is contemplated that by placing the knee in flexion during the subassembly installation procedure, the surgeon may have improved access to the femur box cavity 280, especially when compared to other hinge knee endoprosthetic that require that the hinge component be secured to the tibial component 105. The tibial component 105 can be more obstructed by the patient's soft tissue by this stage. In certain exemplary embodiments, the self-locking taper angle is eight degrees, but different tapers, such as taper angles between and including 8 to 12 degrees can be used.

The femur fastener 210 can be manufactured from any clinically proven biocompatible material. Such materials include but are not necessarily limited to cobalt chromium molybdenum alloys and titanium alloys. It will be appreciated that the femur fastener 210 can comprise any device configured to fixedly engage the femur box 80 to the femoral component 205. Such devices may include but are not necessarily limited to pins with protruding elements, pins with negative elements configured to receive protruding elements from the femur box 80 or other interlocking assembly, bolts, rivets, clamps, interlocking teeth, interlocking hooks, and other projection-receiver locking mechanisms configured to fixedly engage the femur box 80 to the femoral component 205 such that the femur fastener 210 does not receive the load of the femur 200.

Furthermore, and without being bound by theory, it is contemplated that the exemplary embodiments disclosed herein permit the femoral load of the femur (i.e., the weight of the femur and the body above the femur due to gravity) to be primarily transferred from the femur 200 and femoral component 205 to the condylar pads 228, 229 of the meniscal insert 150.

However, in situations where a subset of the femoral load is transferred to the rotating hinge subassembly 10, it is contemplated that the force will be transferred from the femur 200 and femoral component 205 to the femur box 80, transverse hinge pin 50, and head 24 and body 45 of the yoke 40. The bottom of the body 45 of the yoke 40 in turn transferred this load to the tibial component 105, tibia 100, and ultimately the patient's foot when standing or walking. The femoral load can also include the torsional load that the tibia experiences during normal ambulatory movement. The femoral load is not transferred to the femur fastener 210. Therefore, the femur fastener 210 can be shorter than securing fasteners configured to align axially with the tibial rotational axis. In exemplary embodiments, the length of the femur fastener 210 can be about 14 mm for example. Other compatible femur fasteners 210 can have lengths between a range of about 10 mm and about 18 mm.

The transferring of the femoral load through components other than a femur fastener 210 that is configured to mechanically connect the rotating hinge subassembly 10 to the femoral component 205 may prolong the useful life of the endoprosthesis over conventional models in addition to facilitating installation of the endoprosthesis. The fact that the femur fastener 210 can be a single femur fastener 210 in some embodiments, and the fact that the femur fastener 210 can be inserted through the femur box 80 into the femoral component 205 may contribute to an overall reduction in the time required to carry out the surgical procedure.

Femoral Component

In embodiments, the femoral component 205 is provided with features for improved joint function. A femur stem 155 is typically inserted into a femoral bore to seat the femoral component 205 on the resected femoral condyles. The femoral component 205 is configured to provide flexion greater than 120 degrees. The femoral component 205 can be configured to provide uninterrupted patellar kinematics. In certain exemplary embodiments, a femur box cavity 280 (FIG. 2A) of about 16 to about 18 mm in length is provided for the femur box 80 of the rotating hinge subassembly 10. The femoral component 205 can be configured to be compatible with existing revision components, such as femoral augments, offset adapters, and modular stems as well as the modular extension stop 30.

In certain embodiments, both condyles 218, 219 can have a constant radius of curvature. Thus, a ball and socket functionality can be provided on both sides. In other embodiments, the condyles 218, 219 can comprise multiple radii and thereby define a J-curve.

Tibial Component

Referring to FIG. 4, the tibial component 105 includes a conforming axial bore 104 for receiving the tibial axial post 20.

The meniscal insert 150 can conform to mobile bearing features. The proximal/superior side of the tibial base portion 101 is provided with structural features to capture the meniscal insert 150 in a mobile bearing relationship, such as hook features as described further in reference to FIGS. 3A and 3B.

Methods of Use

In operation, the rotating hinge knee endoprosthetic implant assembly 1 of the present disclosure is designed to allow for distal fixation of the rotating hinge subassembly 10 using an anterior approach.

The rotating hinge subassembly 10 can be configured to be pre-assembled, as shown in FIG. 2B, rather than preassembled with the femur or tibia as in prior systems.

After the femoral component 205 and tibial component 105 are implanted, the rotating hinge subassembly 10 is inserted and rotated into place in the femoral component 205. A single tapered-head femur fastener 210 can then be inserted to connect the hinge subassembly 10 to the femoral component 205.

As can be seen from the foregoing descriptions, the exemplary embodiments of the invention have various advantages over prior implants and methods, including but not limited to the following.

The rotating hinge subassembly 10 is configured to be pre-assembled by itself, rather than assembled with the femoral component or tibial component as in prior systems. The transverse hinge pin 50 has a single point of insertion. After the femoral component 205 and tibial component 105 are implanted, the tibial axial post 20 of the rotating hinge subassembly 10 is inserted into the axial bore 104 of the tibial component 105. The rotating hinge subassembly 10 can then be rotated into place to have the femur box 80 fit into femur box cavity 280 of the femoral component 205. This configuration permits a large post length L of the tibial axial post 20 without the need for excessive distraction of the femoral component 205 and tibial component 105 of the rotating hinge knee endoprosthetic implant assembly 1, which in turn permits conservation of more surrounding soft tissue. Preserving surrounding soft tissue can contribute to faster recovery times. Exemplary yokes 40 may have a post length L of about 40 mm to about 70 mm. Additionally, the implant configuration enables surgical methods that require only one incision to install the rotating hinged knee endoprosthetic implant 1, since lateral placement of a hinge pin is eliminated.

Without being bound by theory, it is contemplated that certain exemplary embodiments disclosed herein, coupled with surgical practice, can constrain five of the six directions of movement of the tibial component 105 and the femoral component 205, thereby permitting the surgeon to pre-align the tibial component 105 and the femoral component 205 along one plane of motion to facilitate installation. It will be appreciated that in practice, the femur 200 and the tibia 100 each comprise six fundamental directions of movement. That is, all movement of the femur 200 and the tibia 100 (and therefore any components disposed on the femur or tibia) can be reduced to the sum of movement along the six fundamental directions of movement.

As indicated in FIG. 11A (and prior to the installation of the rotating hinge subassembly 10), the femoral component 205 and tibial component 105 can move relative to each other according to six fundamental directions of movement. That is, the femoral component 205 can move relative to the tibial component 105 (and vice versa) transversely (i.e., along a transverse plane (see 198, FIG. 2D) in an anterior-posterior direction x, in a rotational direction a around the anterior-posterior axis A-P, in a transverse medial-lateral direction y, in a rotational direction b around the transverse medial-lateral axis M-L, in an upward and downward direction z along a parasagittal plane, and in a rotational direction c around the parasagittal upward-downward axis U-D.

An exemplary method comprises placing the tibial axial post 20 of the yoke 40 into the axial bore 104 of the tibial stem 102 while the knee is in flexion. The practice of aligning the femur box cavity 280 of the femoral component 205 with the femur box 80 effectively constrains five of the six fundamental directions of movement of the femoral component 205 relative to the tibial component 105 to the extent that the femoral component 205 is typically moveable primarily upwardly and downwardly along a parasagittal plane z. Any movement in the anterior-posterior direction x, or around the medial-lateral axis on the transverse plane b is thought to be minimal if present. Hands and instrumentation may be used to restrain the range of motion of the femur and tibial to align the femur box cavity 280 of the femoral component 205 with the femur box 80 of the rotating hinge subassembly 10.

FIG. 11B depicts the rotating hinge subassembly 10 in the engaged position. That is, the first joint element has been disposed in the femoral component and engages the femoral component in a projection-receiver locking manner.

In embodiments, the modular extension stop 30 is configured for interaction with the femoral component 205. The modular extension stop 30 can be provided with variable sizing and configured to snap fit into the body 45 of the yoke 40, enabling inventory reduction. The variable sizing of the modular extension stop 30 can also be selected to correspond to specific patient anatomy (e.g., the modular extension stop 30 can be custom made based on preoperative images taken during a preoperative planning stage, or the modular extension stop 30 can be selected from an existing selection of modular extension stops 30 that are provided in a kit). Such modular extension stops 30 can further be selected to address specific defects in the patient's anatomy and to prevent recurvatum. The modular extension stop 30 has locking tabs 32A, 32B extending from a bottom of the modular extension stop 30, the locking tabs 32A, 32B extend into the snap fit pockets 29 of the extension stop portion 28 of the yoke 40 when the modular extension stop 30 is in an installed position.

One tapered-head femur fastener 210 connects the rotating hinge subassembly 10 to the femoral component 205. The use of a single fastener simplifies the assembly and reduces the risk of components loosening from one another during use.

The components of the rotating hinge knee endoprosthetic implant assembly 1 can be provided in the form of a surgical kit. The components of the kit are preferably arranged in a convenient format, such as in a surgical tray or case. However, the kit components do not have to be packaged or delivered together, provided that they are assembled or collected together in the operating room for use at the time of surgery. Exemplary kits may include nine rotating hinge subassemblies 10. In certain exemplary kits, exemplary yokes 40 may have tibial axial posts 20 having one of three lengths L. Such exemplary yokes 40 may have a body 45 having one of three sets of dimensions. The nine rotating hinge subassemblies 10 represent the permutations and combinations of the tibial axial post length L and the dimensions of the body of the tibial yoke 40. Each rotating hinge subassembly 10 may desirably have different offset distance D and post length L from the other rotating hinge subassemblies 10 in the kit.

An exemplary kit can include any suitable embodiment of a rotating hinge subassembly 10, variations of the rotating hinge subassemblies 10 described herein, and any other rotating hinge subassemblies 10 according to an embodiment. While it is contemplated that an exemplary kit may further include one or more modular extension stops 30, etc. one or more tibial components 105, and one or more femoral components 205, it will be appreciated that certain kits may lack some or all of these elements. Any suitable embodiment of a modular extension stop 30, variations of the modular extension stops 30 described herein, and any other modular extension stop 30 according to an embodiment are considered to be within the scope of this disclosure. Any suitable embodiment of a tibial component 105, variations of the tibial components 105 described herein, and any other tibial component 105 according to an embodiment are considered to be within the scope of this disclosure. Any suitable embodiment of a femoral component 205, variations of the femoral components 205 described herein, and any other femoral component 205 according to an embodiment are considered to be within the scope of this disclosure.

Selection of a suitable number or type of rotating hinge subassembly 10, modular extension stop 30, tibial component 105, and femoral component 205, to include in a kit according to a particular embodiment can be based on various considerations, such as the procedure intended to be performed using the components included in the kit.

FIG. 12 depicts a cross-sectional lateral view of an exemplary embodiment comprising a different modular extension stop 30. The cross-sectional view is taken from a parasagittal plane that bisects the rotating hinge knee endoprosthetic implant assembly 1 vertically. As can be seen, at zero degrees flexion, the patella groove 115 of the femoral component 205 is sized and configured to abut against an extension stop 30. In this manner, the rotating hinge knee endoprosthetic implant assembly 1 cannot move into hyperextension or beyond a built-in degree of hyperextension. In the exemplary embodiment of FIG. 12, the femur fastening mechanism (i.e., the femur fastener 210 in the depicted embodiment) is non-axially aligned with the tibial axis of rotation A when the rotating hinge knee endoprosthetic implant assembly 1 is in full extension. That is, the femur fastener's central axis F is not aligned with the tibial axis of rotation A. It is contemplated that this non-alignment when the knee is in extension may further reduce the transfer of any torsional forces from the femur 200 into the femur fastener mechanism and thereby reduce the incidence of wear of the femur fastener mechanism during normal use.

FIG. 13 is a schematic perspective view of anatomical planes relative to a person 600. A transverse plane 198 is shown extending transversely through the person 600. It will be appreciated that although the transverse plane 198 is shown bisecting the person horizontally at the person's midpoint, a transverse plane 198 is an imaginary plane that can be imagined to exist horizontally anywhere on the person 600 between a lateral point that is distally disposed from a medial point along the shortest possible line. In this manner, a transverse plane 198 can be said to divide a person 600 into an upper portion and a lower portion. A sagittal plane 400 is shown bisecting the person 600 vertically in an anteroposterior manner through the midpoint. It will be appreciated that although the sagittal plane 400 is shown bisecting the person, a sagittal plane 400 is an imaginary plane that can be imagined to exist vertically anywhere on the person 600 between an anterior point that is distally disposed from a posterior point along the shortest possible line. In this manner, a sagittal plane 400 divides a person into a left portion and a right portion. A sagittal plane 400 that is not disposed at the median of the person 600 is generally known as a parasagittal plane (see z, FIG. 11A) A coronal plane 500 is shown bisecting the person 600 vertically in a mediolateral manner through the midpoint. It will be appreciated that although the coronal plane 500 is shown bisecting the person, a coronal plane 500 is an imaginary plane that can be imagined to exist vertically anywhere on the person 600 between a lateral point that is distally disposed from a medial point along the shortest possible line. In this manner, a coronal plane 500 divides a person into a front portion and a back portion.

An exemplary knee joint endoprosthesis comprises: a tibial component, a femoral component, and a rotating hinge subassembly configured to couple to the femoral component while having a portion being configured to be disposed in the tibial component, while being rotatable around a rotational axis in the tibial component, the rotating hinge subassembly comprising: a first joint element and a second joint element coupled with the first joint element to be rotatable around the rotational axis, the first joint element defining: an engaged position, wherein the first joint element is disposed in the femoral component and engages the femoral component in a projection-receiver locking manner, and a disengaged position, wherein the first joint element is fully separated from the femoral component, wherein: the first joint element defines an engagement direction, wherein the first joint element is movable relative to the femoral component for transferring the first joint element from the disengaged position into the engaged position, the engagement direction runs transversely to the rotational axis, and projection and receiving elements of the projection-receiver locking manner extends non-parallelly to the engagement direction.

An exemplary rotating hinge subassembly comprises: a tibial yoke comprising: a head defining a transverse hinge bore extending through a first lateral side and a second lateral side of the head, a tibial axial post extending away from the head, and a body member disposed between the head and the tibial axial post, the body member separating the head from the tibial axial post by an offset distance; a femur box comprising: a first arm and a second arm extending posteriorly from a main body portion, the main body portion defining a femur box securing bore extending through the main body portion, wherein the first arm and the second arm respectively define axially aligned first and second bores, wherein the first arm is adjacently disposed to the first lateral side and the second arm is adjacently disposed to the second lateral side such that the first bore and the second bore axially align with the transverse hinge bore; a transverse hinge pin extending through the transverse hinge bore and into the first bore and the second bore.

An exemplary rotating hinge subassembly comprises: a tibial yoke comprising: a head defining a transverse hinge bore extending through a first lateral side and a second lateral side of the head, a tibial axial post extending away from the head, and a body member disposed between the head and the tibial axial post, the body member separating the head from the tibial axial post by an offset distance; a femur box comprising: a first arm and a second arm extending posteriorly from a main body portion, the main body portion defining a femur box securing bore extending through the main body portion, wherein the first arm and the second arm respectively define axially aligned first and second bores, wherein the first arm is adjacently disposed to the first lateral side and the second arm is adjacently disposed to the second lateral side such that the first bore and the second bore axially align with the transverse hinge bore; a transverse hinge pin extending through the transverse hinge bore and into the first bore and the second bore, wherein the transverse hinge pin does not extend beyond the arms of the femur box.

An exemplary knee joint endoprosthesis comprises: a tibial component and a femoral component, the femoral component having areas defining a femoral receiving bore; a preassembled rotating hinge subassembly configured to couple the femoral component and the tibial component to be rotatable around a rotational axis; the preassembled rotating hinge subassembly comprising: a first joint element and a second joint element coupled with the first joint element to be rotatable around the rotational axis, the first joint element having areas defining a first receiving bore, the first joint element defining: an engaged position, wherein the first joint element is disposed in the femoral component such that the first receiving bore and the femoral receiving bore align, and a fastener extends through the first receiving bore and the femoral receiving bore to thereby engage the femoral component, and a disengaged position, wherein the first joint element is fully separated from the femoral component, wherein: the first joint element defines an engagement direction, the first joint element is movable relative to the femoral component for transferring the first joint element from the disengaged position into the engaged position, the engagement direction runs transversely to the rotational axis, and the femoral bore extends in a non-parallel manner to the engagement direction.

In such an exemplary embodiment, the first joint element can be a femur box. Such an exemplary embodiment can further comprise an extension stop.

An exemplary rotating hinge knee endoprosthetic implant assembly comprises: a rotating hinge subassembly having a femur box configured to hingedly articulate around a tibial yoke via a transversely extending hinge pin, the tibial yoke comprising: a body member having a head disposed at a first end, and a tibial axial post extending from a second end of the body member, the second end of the body member being distally disposed from the first end of the body member, wherein the femur box is configured to be mechanically engaged to a femoral component via a femur fastener, wherein the femur fastener is non-axially aligned with a tibial axis of rotation when the knee is in flexion (e.g., when the rotating hinge knee endoprosthetic implant is hingedly rotating around the transverse pin at a flexion angle greater than 0 degrees) or extension (e.g., when the rotating hinge knee endoprosthetic implant is hingedly rotating around the transverse pin at a flexion angle of 0 degrees or less), and wherein the transversely extending hinge pin of the rotating hinge subassembly does not mechanically engage the femoral component in an installed configuration.

An exemplary endoprosthetic tibial yoke for a rotating hinge subassembly comprises: a body member having a head disposed at a first end of the body member; and a tibial axial post extending from a second end of the body member, the second end being distally disposed from the first end, the head defining a transverse hinge bore extending through a first lateral side and a second lateral side of the head, the transverse hinge bore defining a transverse axis extending transversely through the transverse hinge bore, the tibial axial post defining a midline axis extending longitudinally through the tibial axial post, wherein a reference axis extends substantially orthogonally through the transverse axis and substantially parallelly to the midline axis, and wherein an offset distance separates the midline axis from the reference axis.

In an exemplary embodiment of the tibial yoke, the tibial axial post, the body member, and the head are a single contiguous structure.

In an exemplary embodiment of the tibial yoke, the tibial axial post further comprises a distal end disposed distally from the second end of the body member. In such an exemplary embodiment, the distal end can have a shape selected from the group consisting essentially of a convex shape, a chamfered shape, a convex conical shape, a convex hemispherical shape, and a convex frustoconical shape.

In an exemplary embodiment of the tibial yoke, the offset distance is selected from a range of distances consisting essentially of about 12 mm to about 15 mm.

In an exemplary embodiment of the tibial yoke, the tibial axial post further comprises a post length, and wherein the post length has a value of about 40 mm to about 70 mm.

An exemplary tibial yoke may further comprise an extension stop portion extending between the head and the tibial axial post, wherein the extension stop portion comprises snap-fit pockets. In such an exemplary embodiment, the tibial yoke may further comprise a modular extension stop having locking tabs extending from a bottom of the modular extension stop, the locking tabs being configured to extend into the snap fit pockets of the extension stop portion of the yoke when the modular extension stop is in an installed position.

An exemplary rotating hinge subassembly comprises: a tibial yoke comprising: a head defining a transverse hinge bore extending through a first lateral side and a second lateral side of the head, a tibial axial post extending away from the head, and a body member disposed between the head and the tibial axial post, the body member separating the head from the tibial axial post by an offset distance; a femur box comprising: a first arm and a second arm extending posteriorly from a main body portion, the main body portion having a femoral fastening mechanism configured to engage a transverse surface of a femur box cavity of a femoral component of a knee joint endoprosthesis, wherein the first arm and the second arm respectively define axially aligned first and second arm bores, wherein the first arm is adjacently disposed to the first lateral side and the second arm is adjacently disposed to the second lateral side such that the first arm bore and the second arm bore axially align with the transverse hinge bore; a transverse hinge pin extending through the transverse hinge bore and into the first arm bore and the second arm bore.

In an exemplary embodiment of the rotating hinge subassembly the rotating hinge subassembly is preassembled.

In an exemplary embodiment of the rotating hinge subassembly the first arm and the second arm are flushly disposed against the lateral sides of the head.

In an exemplary embodiment of the rotating hinge subassembly the transverse hinge pin does not extend beyond the first and second arms of the femur box.

In an exemplary embodiment of the rotating hinge subassembly, the rotating hinge assembly may further comprise a bearing disposed between a first lateral side of the head and the first arm.

In an exemplary embodiment of the rotating hinge subassembly the femur box is made from a material from a group of materials consisting essentially of a cobalt chrome molybdenum alloy, a titanium allow, a zirconia toughened alumina ceramic, a ceramic material, an ultrahigh molecular weight polyethylene, a polyether ether ketone, combinations thereof, and other biocompatible clinically proven articulating materials.

In an exemplary embodiment of the rotating hinge subassembly the femoral fastening mechanism is selected from the group consisting essentially of a femur box securing bore configured to receive a femur fastener, a projection, a receiver, multiple projections, multiple receivers, a magnet, a clamp, a hook, a lip, a bonding agent, an adhesive, and combinations thereof.

An exemplary rotating hinge subassembly comprises: a tibial yoke comprising: a body member having a head disposed at a first end, and a tibial axial post extending from a second end of the body member, the second end being distally disposed from the first end, the head defining a transverse hinge bore extending through a first lateral side and a second lateral side of the head, the transverse hinge bore defining a transverse axis extending transversely through the transverse hinge bore, the tibial axial post defining a midline axis extending longitudinally through the tibial axial post, wherein a reference axis extends orthogonally through the transverse axis and parallelly to the midline axis, and wherein an offset distance separates the midline axis from the reference axis; a femur box comprising: a first arm extending posteriorly from a main body portion, a second arm extending posteriorly from the main body portion, the second arm opposing the first arm, the main body portion defining a femoral fastening mechanism configured to engage a transverse surface of a femur box cavity of a femoral component of a knee joint endoprosthesis, wherein the first arm defines a first bore, wherein the second arm defines a second bore, wherein the first bore axially aligns with the second bore, wherein the first arm is adjacently disposed to the first lateral side and the second arm is adjacently disposed to the second lateral side such that the first bore and the second bore axially align with the transverse hinge bore; and a transverse hinge pin extending through the transverse hinge bore and into the first bore and the second bore to thereby hingedly engage the femur box to the head of the yoke.

An exemplary knee joint endoprosthesis comprises: a tibial component; a femoral component; and a rotating hinge subassembly having a first joint element configured to be coupled to the femoral component while having a second joint element being configured to be disposed in the tibial component, while being rotatable around a rotational axis in the tibial component, the rotating hinge subassembly comprising: a first joint element and a second joint element hingedly coupled to the first joint element, the first joint element defining: an engaged position, wherein the first joint element is disposed in the femoral component and engages the femoral component in a locking manner, and a disengaged position, wherein the first joint element is fully separated from the femoral component, wherein: the first joint element defines an engagement direction, wherein the first joint element is movable relative to the femoral component for transferring the first joint element from the disengaged position into the engaged position, and the engagement direction runs sagittally to the rotational axis.

In an exemplary knee joint endoprosthesis, the locking manner is a projection-receiver locking manner and wherein projection and receiving elements of the projection-receiver locking manner are disposed coplanar with a parasagittal plane extend non-parallelly to the engagement direction. In such an exemplary knee joint endoprosthesis, the projection-receiver locking manner may comprise: the first joint element having areas defining a first receiving bore, and the femoral component having areas defining a femoral receiving bore, wherein the first receiving bore and the femoral receiving bore align, and a fastener extends through the first receiving bore and the femoral receiving bore.

In an exemplary knee joint endoprosthesis, the parasagittal plane extends through a midpoint of the femoral component.

In an exemplary knee joint endoprosthesis, the locking manner is a bonding or magnetic locking manner.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. A rotating hinge knee endoprosthetic implant assembly comprising:
a rotating hinge subassembly having a femur box configured to hingedly articulate around a tibial yoke via a transversely extending hinge pin, the tibial yoke comprising:
a body member having a head disposed at a first end, and
a tibial axial post extending from a second end of the body member,
the second end of the body member being distally disposed from the first end of the body member,
wherein the femur box is configured to be mechanically engaged to a femoral component via a femur fastener,
wherein the femur fastener is non-axially aligned with a tibial axis of rotation when the knee is in flexion or extension, and wherein the transversely extending hinge pin of the rotating hinge subassembly does not mechanically engage the femoral component in an installed configuration.

2. An endoprosthetic tibial yoke for a rotating hinge subassembly comprising:
a body member having a head disposed at a first end of the body member; and
a tibial axial post extending from a second end of the body member, the second end being distally disposed from the first end,
the head defining a transverse hinge bore extending through a first lateral side and a second lateral side of the head,
the transverse hinge bore defining a transverse axis extending transversely through the transverse hinge bore,
the tibial axial post defining a midline axis extending longitudinally through the tibial axial post,
wherein a reference axis extends substantially orthogonally through the transverse axis and substantially parallelly to the midline axis, and
wherein an offset distance separates the midline axis from the reference axis.

3. The tibial yoke of claim 2, wherein the tibial axial post, the body member, and the head are a single contiguous structure.

4. The tibial yoke of claim 2, wherein the offset distance is selected from a range of distances consisting essentially of: about 12 mm to about 15 mm.

5. The tibial yoke of claim 2, wherein the tibial axial post further comprises a post length, and wherein the post length has a value of about 40 mm to about 70 mm.

6. The tibial yoke of claim 2 further comprising an extension stop portion extending between the head and the tibial axial post, wherein the extension stop portion comprises snap-fit pockets.

7. The tibial yoke of claim 6 further comprising a modular extension stop having locking tabs extending from a bottom of the modular extension stop, the locking tabs being configured to extend into the snap fit pockets of the extension stop portion of the yoke when the modular extension stop is in an installed position.

8. A rotating hinge subassembly comprising:
a tibial yoke comprising:
a head defining a transverse hinge bore extending through a first lateral side and a second lateral side of the head,
a tibial axial post extending away from the head, and
a body member disposed between the head and the tibial axial post, the body member separating the head from the tibial axial post by an offset distance;
a femur box comprising:
a first arm and a second arm extending posteriorly from a main body portion,
the main body portion having a femoral fastening mechanism configured to engage a transverse surface of a femur box cavity of a femoral component of a knee joint endoprosthesis,
wherein the first arm and the second arm respectively define axially aligned first and second arm bores,
wherein the first arm is adjacently disposed to the first lateral side and the second arm is adjacently disposed to the second lateral side such that the first arm bore and the second arm bore axially align with the transverse hinge bore;
a transverse hinge pin extending through the transverse hinge bore and into the first arm bore and the second arm bore.

9. The rotating hinge subassembly of claim 8, wherein the rotating hinge subassembly is preassembled.

10. The rotating hinge subassembly of claim 8, wherein the first arm and the second arm are flushly disposed against the lateral sides of the head.

11. The rotating hinge subassembly of claim 8, wherein the transverse hinge pin does not extend beyond the first and second arms of the femur box.

12. The rotating hinge subassembly of claim 8 further comprising a bearing disposed between a first lateral side of the head and the first arm.

13. The rotating hinge subassembly of claim 8, wherein the femur box is made from a material from a group of materials consisting essentially of: a cobalt chrome molybdenum alloy, a titanium allow, a zirconia toughened alumina ceramic, a ceramic material, an ultrahigh molecular weight polyethylene, a polyether ether ketone, combinations thereof, and other biocompatible clinically proven articulating materials.

14. The rotating hinge subassembly of claim 8, wherein the femoral fastening mechanism is selected from the group consisting essentially of: a femur box securing bore configured to receive a femur fastener, a projection, a receiver, multiple projections, multiple receivers, a magnet, a clamp, a hook, a lip, a bonding agent, an adhesive, and combinations thereof.

15. A rotating hinge subassembly comprising:
a tibial yoke comprising:
a body member having a head disposed at a first end, and
a tibial axial post extending from a second end of the body member, the second end being distally disposed from the first end,
the head defining a transverse hinge bore extending through a first lateral side and a second lateral side of the head,
the transverse hinge bore defining a transverse axis extending transversely through the transverse hinge bore,
the tibial axial post defining a midline axis extending longitudinally through the tibial axial post,
wherein a reference axis extends orthogonally through the transverse axis and parallelly to the midline axis, and
wherein an offset distance separates the midline axis from the reference axis;
a femur box comprising:
a first arm extending posteriorly from a main body portion,
a second arm extending posteriorly from the main body portion,
the second arm opposing the first arm,
the main body portion defining a femoral fastening mechanism configured to engage a transverse surface of a femur box cavity of a femoral component of a knee joint endoprosthesis,
wherein the first arm defines a first bore,
wherein the second arm defines a second bore,
wherein the first bore axially aligns with the second bore,
wherein the first arm is adjacently disposed to the first lateral side and the second arm is adjacently disposed to the second lateral side such that the first bore and the second bore axially align with the transverse hinge bore; and
a transverse hinge pin extending through the transverse hinge bore and into the first bore and the second bore to thereby hingedly engage the femur box to the head of the yoke.

16. A knee joint endoprosthesis comprising:
a tibial component;
a femoral component; and
a rotating hinge subassembly having a first joint element configured to be coupled to the femoral component while having a second joint element being configured to be disposed in the tibial component, while being rotatable around a rotational axis in the tibial component, the rotating hinge subassembly comprising:
a first joint element and a second joint element hingedly coupled to the first joint element,
the first joint element defining:
an engaged position, wherein the first joint element is disposed in the femoral component and engages the femoral component in a locking manner, and
a disengaged position, wherein the first joint element is fully separated from the femoral component,
wherein:
the first joint element defines an engagement direction, wherein the first joint element is movable relative to the femoral component for transferring the first joint element from the disengaged position into the engaged position, and
the engagement direction runs sagittally to the rotational axis.

17. The knee joint endoprosthesis of claim 16, wherein the locking manner is a projection-receiver locking manner and wherein projection and receiving elements of the projection-receiver locking manner are disposed coplanar with a parasagittal plane extending non-parallelly to the engagement direction.

18. The knee joint endoprosthesis of claim 17, wherein the projection-receiver locking manner comprises:
the first joint element having areas defining a first receiving bore, and
the femoral component having areas defining a femoral receiving bore,
wherein the first receiving bore and the femoral receiving bore align, and a fastener extends through the first receiving bore and the femoral receiving bore.

19. The knee joint endoprosthesis of claim 17 wherein the parasagittal plane extends through a midpoint of the femoral component.

20. The knee joint endoprosthesis of claim 16, wherein the locking manner is a bonding or magnetic locking manner.
